(19) United States
(12) Patent Application Publication
(10) Pub. No.: US 2025/0041277 A1
(43) Pub. Date: Feb. 6, 2025

(54) METHODS AND DOSING REGIMENS COMPRISING A CDK2 INHIBITOR AND A CDK4 INHIBITOR FOR TREATING CANCER

(71) Applicant: PFIZER INC., NEW YORK, NY (US)

(72) Inventors: Lars ANDERS, San Diego, CA (US); Jerry LI, San Diego, CA (US); Tun Tun LIN, San Diego, CA (US); Ping WEI, San Diego, CA (US); Jing YANG, Bellevue, WA (US); Todd Lee VANARSDALE, San Diego, CA (US)

(73) Assignee: PFIZER INC., NEW YORK, NY (US)

(21) Appl. No.: 18/715,085

(22) PCT Filed: Dec. 1, 2022
(86) PCT No.: PCT/IB2022/061652

§ 371 Date: May 30, 2024

Related U.S. Application Data
(60) Provisional application No. 63/285,457, filed on Dec. 2, 2021.

(51) Int. Cl. A61K 31/4155; A61K 31/4196; A61K 31/506; A61K 31/565; A61P 35/00

(52) U.S. Cl. CPC A61K 31/4155; A61K 31/4196; A61K 31/506; A61K 31/565; A61P 35/00

(57) ABSTRACT

This disclosure relates to combination therapies for use in treating cancer, comprising a CDK2 inhibitor of Formula (I) and a selective CDK4 inhibitor of Formula (II), each as further described herein, optionally in further combination with an additional anti-cancer agent.

### BACKGROUND

### Field of the Disclosure

The present disclosure relates to methods and combination therapies useful for treating cancer in a subject in need thereof. In particular, the disclosure relates to methods and combinations comprising a cyclin dependent kinase 2 (CDK2) inhibitor, such as (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (hereinafter PF-07104091) or a monohydrate thereof, in combination with a selective cyclin dependent kinase 4 (CDK4) inhibitor, such as 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (hereinafter PF-07220060) or a pharmaceutically acceptable salt thereof, wherein the combination is optionally in further combination with an additional anti-cancer agent such as an endocrine therapy agent. The disclosure also relates to associated dosage regimens, uses and pharmaceutical compositions.

### Description of the Related Art

Cyclin-dependent kinases (CDKs) are important cellular enzymes that perform essential functions in regulating eukaryotic cell division and proliferation. CDK inhibitors may be useful for the treatment of proliferative disorders, including cancer.

Overexpression of CDK2 is associated with abnormal regulation of the cell-cycle. The cyclin E/CDK2 complex plays and important role in regulation of the G1/S transition, histone biosynthesis and centrosome duplication. Progressive phosphorylation of retinoblastoma (RB) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al. The history and future of targeting cyclin-dependent kinases in cancer therapy, Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol. Cancer Ther. (2012) 11:1488-99; Herrera-Abreu et al., Early Adaptation and Acquired Resistance to CDK4/6 Inhibition in Estrogen Receptor-Positive Breast Cancer, Cancer Res. (2016) 76: 2301-2313). Cyclin E amplification also reportedly contributes to trastuzumab resistance in human epidermal growth factor receptor 2 positive (HER2+) breast cancer. (Scaltriti et al. Cyclin E amplification/overexpression is a mechanism of trastuzumab resistance in HER2+ breast cancer patients, Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Triple Negative Breast Cancer: Clinical and Histological Correlations, Breast Care (2011) 6:273-278; Alexander et al., Cyclin E overexpression as a biomarker for combination treatment strategies in inflammatory breast cancer, Oncotarget (2017) 8: 14897-14911.)

High CCNE1 mRNA expression was found to be associated with relative resistance to palbociclib in a gene expression analysis of the PALOMA-3 trial, suggesting a role for CDK2 inhibition in reducing or overcoming resistance to CDK4/6 inhibition. (Turner et al., Cyclin E1 Expression and Palbociclib Efficacy in Previously Treated Hormone Receptor-Positive Metastatic Breast Cancer, J. Clin. Oncol. (2019) 37:1169-1178). Amplification or overexpression of cyclin E1 (CCNE1) is also associated with poor outcomes in ovarian, gastric, endometrial, and other cancers. (Nakayama et al., Gene amplification CCNE1 is related to poor survival and potential therapeutic target in ovarian cancer, Cancer (2010) 116: 2621-34; Etemadmoghadam et al., Resistance to CDK2 Inhibitors Is Associated with Selection of Polyploid Cells in CCNE1-Amplified Ovarian Cancer, Clin Cancer Res (2013) 19: 5960-71; Au-Yeung et al., Selective Targeting of Cyclin E1-Amplified High-Grade Serous Ovarian Cancer by Cyclin-Dependent Kinase 2 and AKT Inhibition, Clin. Cancer Res. (2017) 23:1862-1874; Ayhan et al., CCNE1 copy-number gain and overexpression identify ovarian clear cell carcinoma with a poor prognosis, Modern Pathology (2017) 30: 297-303; Ooi et al., Gene amplification of CCNE1, CCND1, and CDK6 in gastric cancers detected by multiplex ligation-dependent probe amplification and fluorescence in situ hybridization, Hum Pathol. (2017) 61: 58-67; Noske et al., Detection of CCNE1/URI (19q12) amplification by in situ hybridisation is common in high grade and type II endometrial cancer, Oncotarget (2017) 8: 14794-14805).

CDK4 and CDK6 are important regulators of cell cycle progression at the G1-S checkpoint, which are controlled by D-type cyclins and INK4 endogenous CDK inhibitors, such as p16^{INK4a} (CDKN2A). Dysregulation of the cyclin D-CDK4/6-INK4-retinoblastoma (Rb) pathway has been reported to be associated with development of endocrine therapy resistance.

CDK4/6 inhibition has emerged as a promising strategy for cancer therapy, especially for the treatment of endocrine resistant breast cancer (BC). (Rani, A., et. al., Endocrine Resistance in Hormone Receptor Positive Breast Cancer—From Mechanism to Therapy. Front Endocrinol (Lausanne) 10:245, 2019).

CDK4/6 inhibitors (e.g., palbociclib, abemaciclib, ribociclib) when dosed in combination with endocrine therapy, have significantly improved progression-free survival and/or overall survival for patients with HR-positive/HER2-negative advanced or metastatic breast cancer. (Spring, L. M., et. al., Cyclin-dependent kinase 4 and 6 inhibitors for hormone receptor-positive breast cancer: past, present, and future. Lancet, 395, 817-827, 2020). However, CDK4/6 inhibitors have been associated with dose-limiting hematologic toxicities, primarily neutropenia, and gastrointestinal toxicities. As with other kinase inhibitors, the effectiveness of CDK4/6 inhibitors may be limited over time by the development of primary or acquired resistance.

Emerging data suggest that cyclin D3-CDK6 may be linked to the observed hematologic toxicity. (Malumbres et al., Mammalian Cells Cycle without the D-type Cyclin-Dependent Kinases Cdk4 and Cdk6, (2004) Cell 118(4):493-504; Sicinska et al. Essential Role for Cyclin D3 in Granulocyte Colony-Stimulating Factor-Driven Expansion of Neutrophil Granulocytes (2006), Mol. Cell Biol 26(21): 8052-8060; Cooper et al. A unique function for cyclin D3 in early B cell development, (2006), Nat. Immunol. 5(7):489-497). CDK4 has been identified as the singular oncogenic driver in many breast cancers. Accordingly, a CDK4 selective inhibitor may provide an improved safety profile or enhanced overall efficacy due to the potential of higher and/or continuous dosing compared to dual CDK4/6 inhibitors.

The compound (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (hereinafter PF-07104091) is a potent and selective inhibitor of CDK2, having the structure:

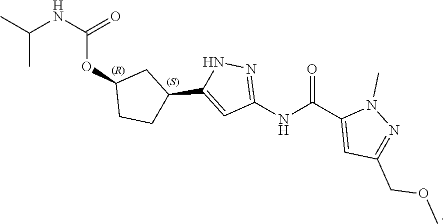

PF-07104091 (Pfizer Inc.) is currently in clinical development for the treatment of certain cancers. Preparation of PF-07104091 is disclosed in International Patent Publication No. WO 2020/157652 and in U.S. Pat. No. 11,014,911, the contents of each which are incorporated herein by reference in their entirety. PF-07104091 also has a name (1R,3S)-3-(3-(3-(methoxymethyl)-1-methyl-1H-pyrazole-5-carboxamido)-1H-pyrazol-5-yl)cyclopentyl isopropylcarbamate, as generated by ChemDraw 20.1.1.

The compound 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (hereinafter PF-07220060) is a potent and selective inhibitor of CDK4, having the structure:

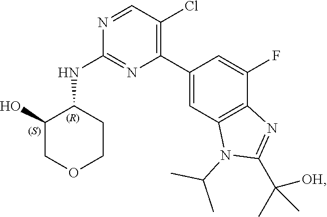

or a pharmaceutically acceptable salt thereof.

PF-07220060 (Pfizer Inc.) is currently in clinical development for the treatment of certain cancers. Preparation of PF-07220060 is disclosed in International Patent Publication No. WO 2019/207463 and U.S. Pat. No. 10,766,884, the contents of each which are incorporated herein by reference in their entirety. PF-07220060 also has a name (3S,4R)-4-((5-chloro-4-(4-fluoro-2-(2-hydroxypropan-2-yl)-1-isopropyl-1H-benzo[d]imidazol-6-yl)pyrimidin-2-yl)amino)tetrahydro-2H-pyran-3-ol, as generated by ChemDraw 20.1.1.

There remains a need for improved therapies for the treatment of cancers. The combinations, methods, dosage regimens and uses disclosed herein are believed to have one or more advantages, such as greater efficacy than treatment with either therapeutic agent alone; potential to reduce drug-drug interactions; potential to enable an improved dosing schedule; potential to reduce side effects; potential to overcome resistance mechanisms and the like.

### BRIEF SUMMARY

The disclosure relates to methods, combinations, dosage regimens, uses and pharmaceutical compositions for treating cancer in a subject in need thereof, comprising a selective CDK2 inhibitor of Formula (I) or a pharmaceutically acceptable salt or solvate thereof, and a selective CDK4 inhibitor, such as a compound of Formula (II) or a pharmaceutically acceptable salt thereof.

In one aspect, the disclosure provides a method of treating cancer in a subject in need thereof comprising administering to the subject:
(a) an amount of a compound of Formula (I):

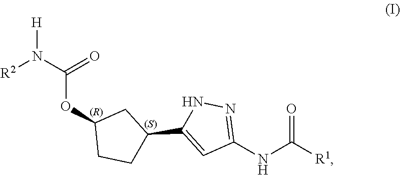

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) an amount of a selective cyclin dependent kinase 4 (CDK4) inhibitor;
wherein the amounts in (a) and (b) together are effective in treating cancer.

In some embodiments of this aspect, the disclosure provides a method further comprising administering to the subject: (c) an amount of an additional anti-cancer agent; wherein the amounts in (a), (b) and (c) together are effective in treating cancer.

In another aspect, the disclosure provides a combination comprising:
(a) a compound of Formula (I):

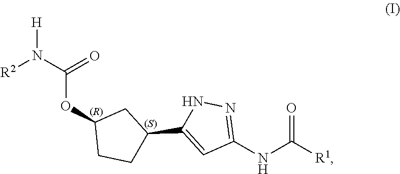

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) a selective cyclin dependent kinase 4 (CDK4) inhibitor;
wherein the combination of (a) and (b) is effective in treating cancer.

In some embodiments of this aspect, the combination further comprises (c) an additional anti-cancer agent; wherein the combination of (a), (b) and (c) is effective in treating cancer.

In another aspect, the disclosure provides a combination for use in treating cancer comprising:
(a) a compound of Formula (I):

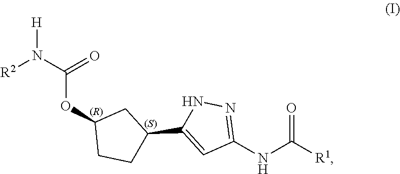

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where
each C₁-C₄ alkyl is optionally substituted by F, OH or C₁-C₄ alkoxy; and
(b) a selective cyclin dependent kinase 4 (CDK4) inhibitor.

In some embodiments of this aspect, the combination for use further comprises (c) an additional anti-cancer agent.

In another aspect, the disclosure provides use of a combination comprising:
(a) a compound of Formula (I):

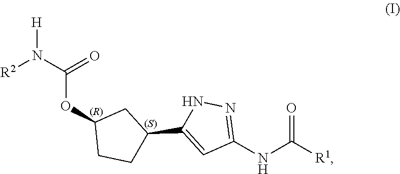

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where
each C₁-C₄ alkyl is optionally substituted by F, OH or C₁-C₄ alkoxy; and
(b) a selective cyclin dependent kinase 4 (CDK4) inhibitor;
wherein use of the combination is effective in treating cancer.

In some embodiments of this aspect, the combination further comprises (c) an additional anti-cancer agent, wherein the use of the combination of (a), (b) and (c) is effective in treating cancer.

In preferred embodiments of each of the methods, combination and uses herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091), a potent inhibitor of CDK2, having the structure:

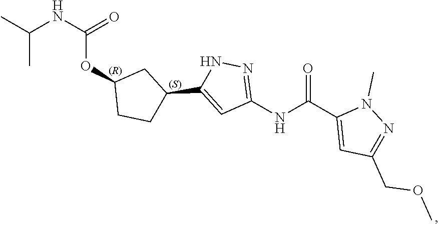

or a monohydrate thereof.

In particularly preferred embodiments of each of the methods, combinations and uses described herein, the compound of Formula (I) is PF-07104091 monohydrate.

In some embodiments of each of the methods, combinations and uses described herein, the combination of the compound of Formula (I) and the selective CDK4 inhibitor is synergistic. In some embodiments of the methods, combinations and uses described herein, the combination of the compound of Formula (I), the CDK4 inhibitor, and the additional anti-cancer agent is synergistic.

In some embodiments of each of the methods, combinations and uses described herein, the selective CDK4 inhibitor is a compound of Formula (II):

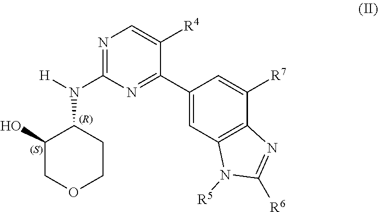

or a pharmaceutically acceptable salt thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl.

In preferred embodiments, the compound of Formula (II) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (PF-07220060), a potent and selective inhibitor of CDK4, having the structure:

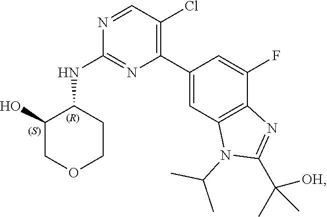

or a pharmaceutically acceptable salt thereof.

In preferred embodiments of each of the methods, combinations and uses described herein, the compound of Formula (I) is PF-07104091 or a pharmaceutically acceptable solvate thereof, and the compound of Formula (II) is PF-07220060 or a pharmaceutically acceptable salt thereof. In particularly preferred embodiments of each of the methods, combinations and uses described herein, the compound of Formula (I) is PF-07104091 monohydrate, and the compound of Formula (II) is PF-07220060.

In some embodiments of each of the methods, combinations and uses described herein, the disclosure further comprises one or more additional anti-cancer agents. In preferred embodiments, the cancer is breast cancer (including HR+/HER2− breast cancer) and the additional anti-cancer agent is an endocrine therapeutic agent. In some such embodiments, the endocrine therapeutic agent is an aromatase inhibitor, a selective estrogen receptor degrader (SERD), or a selective estrogen receptor modulator (SERM). In preferred embodiments, the endocrine therapeutic agent is letrozole or fulvestrant.

Embodiments of each of the methods, combinations and uses described herein may be combined with one or more other embodiments t, provided such embodiments are not inconsistent with each other.

### DETAILED DESCRIPTION

The present disclosure may be understood more readily by reference to the following detailed description of the preferred embodiments of the disclosure and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

E1. A method of treating cancer in a subject in need thereof comprising administering to the subject:
(a) an amount of a compound of Formula (I):

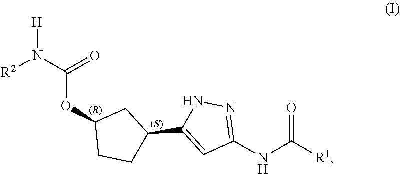

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) an amount of a compound of Formula (II):

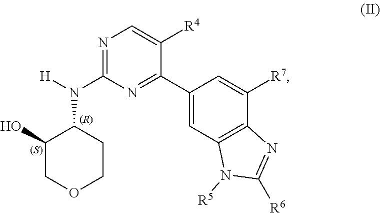

or a pharmaceutically acceptable salt thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl;
wherein the amounts in (a) and (b) together are effective in treating cancer.

E2. The method of embodiment E1, further comprising administering to the subject: (c) an amount of an additional anti-cancer agent; wherein the amounts in (a), (b) and (c) together are effective in treating cancer.

E3. The method of embodiment E1 or E2, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E4. The method of embodiment E2 or E3, wherein the cancer is hormone receptor positive (HR+) breast cancer and the additional anti-cancer agent is an endocrine therapeutic agent selected from the group consisting of an aromatase inhibitor, a selective estrogen receptor modulator (SERM) and a selective estrogen receptor degrader (SERD).

E5. The method of embodiment E4, wherein the endocrine therapeutic agent is letrozole or fulvestrant.

E6. The method of any one of embodiments E1 to E3, wherein the cancer is lung cancer.

E7. The method of embodiment E6, wherein the lung cancer is small cell lung cancer (SCLC).

E8. The method of embodiment E7, wherein the SCLC is characterized by loss of retinoblastoma (RB) function.

E9. The method of embodiment E6, wherein the lung cancer is non-small cell lung cancer (NSCLC).

E10. The method of embodiment E9, wherein the NSCLC is lung squamous cell carcinoma (LUSC) or lung adenocarcinoma (LUAD).

E11. The method of embodiment E10, wherein the NSCLC is KRAS-driven lung adenocarcinoma (LUAD).

E12. The method of any one of embodiments E1 to E11, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091) monohydrate.

E13. The method of embodiment E12, wherein the amount of PF-07104091 is from about 75 mg to about 500 mg BID.

E14. The method of embodiment E13, wherein the amount of PF-07104091 is about 75 mg BID, about 150 mg BID, or about 225 mg BID.

E15. The method of embodiment E12, wherein the amount of PF-07104091 is from about 100 mg to about 500 mg BID.

E16. The method of embodiment E15, wherein the amount of PF-07104091 is from about 150 mg to about 300 mg BID.

E17. The method of embodiment E15 or E16, wherein the amount of PF-07104091 is about 150 mg BID, about 225 mg BID, or about 300 mg BID.

E18. The method of any one of embodiments E1 to E17, wherein the compound of Formula (II) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (PF-07220060), or a pharmaceutically acceptable salt thereof.

E19. The method of embodiment E18, wherein the amount of PF-07220060 is from about 100 mg to about 500 mg BID.

E20. The method of embodiment E19, wherein the amount of PF-07220060 is from about 300 mg to about 500 mg BID.

E21. The method of embodiment E20, wherein the amount of PF-07220060 is about 100 mg BID, about 200 mg BID, or about 300 mg BID.

E22. The method of embodiment E19 or E20, wherein the amount of PF-07220060 is about 300 mg BID or about 400 mg BID.

E23. A combination comprising:
(a) a compound of Formula (I):

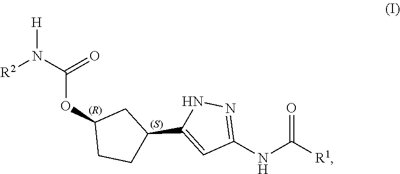

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) a compound of Formula (II):

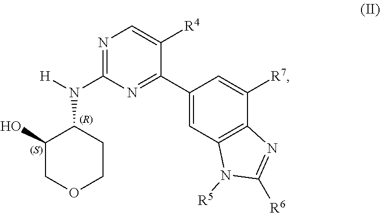

or a pharmaceutically acceptable salt thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl;
wherein the combination of (a) and (b) is effective in treating cancer.

E24. The combination of embodiment E23, further comprising (c) an additional anti-cancer agent; wherein the combination of (a), (b) and (c) is effective in treating cancer.

E25. The combination of embodiment E23 or E24, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E26. The combination of embodiment E24 or E25, wherein the cancer is hormone receptor positive (HR+) breast cancer and the additional anti-cancer agent is an endocrine therapeutic agent selected from the group consisting of an aromatase inhibitor, a selective estrogen receptor modulator (SERM) and a selective estrogen receptor degrader (SERD).

E27. The combination of embodiment E26, wherein the endocrine therapeutic agent is letrozole or fulvestrant.

E28. The combination of any one of embodiments E23 to E25, wherein the cancer is lung cancer.

E29. The combination of embodiment E28, wherein the lung cancer is small cell lung cancer (SCLC).

E30. The combination of embodiment E29, wherein the SCLC is characterized by loss of retinoblastoma (RB) function.

E31. The combination of embodiment E28, wherein the lung cancer is non-small cell lung cancer (NSCLC).

E32. The combination of embodiment E31, wherein the NSCLC is lung squamous cell carcinoma (LUSC) or lung adenocarcinoma (LUAD).

E33. The combination of embodiment E32, wherein the NSCLC is KRAS-driven lung adenocarcinoma (LUAD).

E34. The combination of any one of embodiments E23 to E33, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091) monohydrate.

E35. The combination of embodiment E34, wherein the amount of PF-07104091 is from about 50 mg to about 250 mg BID.

E36. The combination of embodiment E35, wherein the amount of PF-07104091 is from about 75 mg to about 150 mg BID.

E37. The combination of embodiment E35 or E36, wherein the amount of PF-07104091 is about 75 mg BID, about 100 mg BID, about 125 mg BID, or about 150 mg BID.

E38. The combination of embodiment E35, wherein the amount of PF-07104091 is about 75 mg BID, about 150 mg BID, or about 225 mg BID.

E39. The combination of any one of embodiments E23 to E38, wherein the compound of Formula (II) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (PF-07220060), or a pharmaceutically acceptable salt thereof.

E40. The combination of embodiment E39, wherein the amount of PF-07220060 is from about 100 mg to about 500 mg BID.

E41. The combination of embodiment E40, wherein the amount of PF-07220060 is from about 300 mg to about 500 mg BID.

E42. The combination of embodiment E40 or E41, wherein the amount of PF-07220060 is about 300 mg BID, or about 400 mg BID.

E43. The method of embodiment E40, wherein the amount of PF-07220060 is about 100 mg BID, about 200 mg BID, or about 300 mg BID.

E44. A method of treating cancer in a subject in need thereof comprising administering to the subject:
(a) an amount of PF-07104091; and
(b) an amount of PF-07220060, or a pharmaceutically acceptable salt thereof;
wherein the amounts in (a) and (b) together are effective in treating cancer.

E45. A method of treating cancer in a subject in need thereof comprising administering to the subject:
(a) an amount of PF-07104091;
(b) an amount of PF-07220060, or a pharmaceutically acceptable salt thereof; and
(c) an amount of an additional anti-cancer agent;
wherein the amounts in (a), (b) and (c) together are effective in treating cancer.

E46. The method of embodiment E44 or E45, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E47. The method of any one of embodiments E44 to E46, wherein the cancer is hormone receptor positive (HR+), human epidermal growth factor receptor 2 negative (HER2−) breast cancer.

E48. The method of any one of embodiments E45 to E47, wherein and the additional anti-cancer agent is an endocrine therapeutic agent selected from the group consisting of an aromatase inhibitor, a SERM, and a SERD.

E49. The method of embodiment E48, wherein the endocrine therapeutic agent is letrozole or fulvestrant.

E50. The method of any one of embodiments E44 to E46, wherein the cancer is lung cancer.

E51. The method of embodiment E50, wherein the lung cancer is small cell lung cancer (SCLC).

E52. The method of embodiment E51, wherein the SCLC is characterized by loss of retinoblastoma (RB) function.

E53. The method of embodiment E50, wherein the lung cancer is non-small cell lung cancer (NSCLC).

E54. The method of embodiment E53, wherein the NSCLC is lung squamous cell carcinoma (LUSC) or lung adenocarcinoma (LUAD).

E55. The method of embodiment E54, wherein the NSCLC is KRAS-driven lung adenocarcinoma (LUAD).

E56. The method of any one of embodiments E44 to E55, wherein the amount of PF-07104091 is from about 50 mg to about 250 mg BID.

E57. The method of embodiment E56, wherein the amount of PF-07104091 is from about 75 mg to about 150 mg BID.

E58. The method of embodiment E56 or E57, wherein the amount of PF-07104091 is about 75 mg BID, about 100 mg BID, about 125 mg BID, or about 150 mg BID.

E59. The method of embodiment E56 or E57, wherein the amount of PF-07104091 is about 75 mg BID, about 150 mg BID, or about 225 mg BID.

E60. The method of any one of embodiments E44 to E59, wherein the compound of Formula (II) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (PF-07220060), or a pharmaceutically acceptable salt thereof.

E61. The method of embodiment E60, wherein the amount of PF-07220060 is from about 100 mg to about 500 mg BID.

E62. The method of embodiment E61, wherein the amount of PF-07220060 is from about 300 mg to about 500 mg BID.

E63. The method of embodiment E61, wherein the amount of PF-07220060 is about 100 mg BID, about 200 mg BID, or about 300 mg BID.

E64. The method of embodiment E61 or E62, wherein the amount of PF-07220060 is about 300 mg BID, or about 400 mg BID.

E65. The method of any one of embodiments E1 to E22, wherein the cancer is a CDK4/6 inhibitor resistant cancer.

E66. The method of embodiment E65, wherein the CDK4/6 inhibitor is palbociclib.

E67. The method of any one of embodiments E1 to E22, wherein the subject has previously been treated with a CDK4/6 inhibitor.

E68. The method of embodiment E67, wherein the CDK4/6 inhibitor is palbociclib.

### Definitions

As used herein, the singular form “a”, “an”, and “the” include plural references unless indicated otherwise. For example, “a” substituent includes one or more substituents.

The disclosure described herein may be suitably practiced in the absence of any element(s) not specifically disclosed herein. Thus, for example, in each instance herein any of the terms “comprising”, “consisting essentially of”, and “consisting of” may be replaced with either of the other two terms.

The term “about” means having a value falling within an accepted standard of error of the mean, when considered by one of ordinary skill in the art. In some embodiments, the term “about” means within ±10% of the indicated value. For example, a dose of about 150 mg should be understood to mean that the dose may vary between 135 mg and 165 mg.

The terms “cancer”, “cancerous”, or “malignant” refer to or describe the physiological condition in mammals that is typically characterized by unregulated cell growth. As used herein, cancer may refer to any malignant and/or invasive growth or tumor caused by abnormal cell growth, including solid tumors named for the type of cells that form them, as well as cancer of blood, bone marrow, or the lymphatic system. Examples of solid tumors include but not limited to sarcomas and carcinomas. Examples of cancers of the blood include but not limited to leukemias, lymphomas and myeloma. The term cancer includes but is not limited to a primary cancer that originates at a specific site in the body, a metastatic cancer that has spread from the place in which it started to other parts of the body, a recurrence from the original primary cancer after remission, or a second primary cancer that is a new primary cancer in a person with a history of previous cancer of a different type from latter one.

As used herein, “PF-07104091” may refer to PF-07104091 free base and/or PF-07104091 monohydrate. In preferred embodiments, PF-07104091 as referred to herein is PF-07104091 monohydrate.

As used herein, “dose limiting toxicity” (DLT) refers to the dosage, for example of PF-07104091 or another agent described herein, that is contraindicative of a further increase in dosage.

As used herein “maximum tolerated dose” (MTD) refers to the highest dosage of PF-07104091 or another agent described herein that does not cause unacceptable side effects or intolerable toxicities. MTD is estimated using the mTPI based on observed DLT rate.

The terms “patient” or “subject” refer to any single subject for which therapy is desired or that is participating in a clinical trial, epidemiological study or used as a control, including humans and mammalian veterinary patients, e.g., domestic animals such as cattle, horses, dogs, and cats; non-human primates such as monkeys; laboratory animals such as rats, mice, guinea pigs; and captive wild animals such as lions, tigers, and the like. In preferred embodiments, the subject is a human.

In some embodiments, the subject is an adult human subject. In some embodiments, the adult subject is a woman of any menopausal status or a man. In some such embodiments, the subject is a post-menopausal woman or a man. In some such embodiments, the subject is a post-menopausal woman. In some such embodiments, the subject is a pre-menopausal or peri-menopausal woman. In some such embodiments, the subject is a pre-menopausal or peri-menopausal woman treated with a luteinizing hormone-releasing hormone (LHRH) agonist. In some such embodiments, the subject is a man. In some such embodiments, the subject is a man treated with an LHRH agonist. In some embodiments, the subject is a human child between the ages of birth and 18. In some embodiments, the subject is a child between the ages of birth and 15 having a pediatric cancer.

For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C<10% is considered a high anti-tumor activity level, with T/C (%)=median tumor volume of the treated/median tumor volume of the control×100.

The therapeutic effect of the methods, combinations and uses herein, may be defined by reference to any of the following: partial response (PR), complete response (CR), progression free survival (PFS), disease free survival (DFS), duration of response (DoR), overall response rate (ORR), or overall survival (OS). In some embodiments, response to a combination, method or use disclosed herein is any of PR, CR, PFS, DFS, DoR, ORR or OS that is assessed using RECIST v1.1 response criteria (Eisenhauer et al., New response evaluation criteria in solid tumours: Revised RECIST guideline (version 1.1), Eur J of Cancer, 2009; 45(2):228-47).

In some embodiments, the methods, combinations and uses herein relate to neoadjuvant therapy, adjuvant therapy, first-line therapy, second-line therapy, or third-line or later lines of therapy. In each case as further described herein, the cancer may be localized, advanced or metastatic, and the intervention may occur at point along the disease continuum (i.e., at any stage of the cancer).

The treatment regimen for a combination, method or use that is effective to treat cancer in a subject may vary according to factors such as the disease state, age, and weight of the subject, and the ability of the therapy to elicit an anti-cancer response in the subject. While an embodiment of any of the aspects disclosed herein may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-test and the Wilcon on-test.

As used herein, an “effective amount” or “therapeutically effective amount” for use and/or for treating a subject refers to an amount that provides, in single or multiple doses, alone, or in combination with one or more other agents, treatments, protocols, or therapeutic regimens, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences, or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an active agent effective for producing a desired therapeutic effect upon administration to a subject, for example, to stem the growth, or result in the shrinkage, of a cancerous tumor.

The effective amount may vary according to factors such as the disease state, age, sex, and weight of the subject. For prophylactic use, beneficial or desired outcomes may include eliminating or reducing the risk, lessening the severity, or delaying the onset of the disease. For therapeutic use, beneficial or desired outcomes may include reducing the incidence or ameliorating one or more symptoms of the disease, reducing the dose of another medication used to treat the disease, enhancing the efficacy or safety of another medication used to treat the disease, delaying the time to disease progression, or prolonging survival.

As used herein, a “treatment cycle” refers to a period of time comprising administration of one or more agents, with or without rest periods between treatment cycles. A treatment cycle may be continuous, i.e., with no rest period between the treatment cycles. Alternatively, a treatment cycle may be intermittent and include a rest period (i.e., a period of dose interruption of one or more days or weeks off treatment) between treatment cycles. In such instances, administration of another agent during the rest period should not interfere or be detrimental to administration of the agent(s) described herein.

For example, a 21 day or 28 day treatment cycle, with 14 or 21 days on treatment, respectively, followed by a 7 day rest period (i.e., treatment interruption) is an example of an intermittent treatment cycle. Treatment cycles with 2 or 3 weeks on treatment and 1 week off treatment are sometimes referred to as a 2/1-week or 3/1-week treatment cycles, respectively. Alternatively, an intermittent treatment cycle may comprise a 7 day cycle, with 5 days on treatment and 2 days off treatment.

As used herein, the term “ameliorate” refers to any reduction in the extent, severity, frequency, and/or likelihood of a symptom or clinical sign characteristic of a particular disease. “Symptom” refers to any subjective evidence of disease or of a subject's condition.

As used herein, “treat” or “treating” a cancer and/or a cancer-associated disease means to administer a mono- or combination therapy according to the present disclosure to a subject, patient or individual having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (2009)).

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia's (cancers of the blood) generally do not form solid tumors (National Cancer Institute, Dictionary of Cancer Terms).

As used herein, the terms “combination” or “combination therapy” refer to the administration of two or more therapeutic agents of the combination therapy, either as compounds or in the form of a pharmaceutical composition or medicament. The combination therapy may be administered sequentially, concurrently or simultaneously.

When administering a combination therapy of two or more agents, the agents may be administered on the same treatment cycle or using different cycles. In preferred embodiments, PF-07104091 is administered continuously on a 28 day cycle. In preferred embodiments, PF-07220060 is administered continuously on a 28 day cycle. Fulvestrant is typically administered intramuscularly on days 1, 15, 29 of the first treatment cycle and once monthly thereafter. Letrozole is typically administered continuously on a 28 day treatment cycle.

Each therapeutic agent of the methods and combination therapies described herein may be administered as a compound, or in a pharmaceutical composition (also referred to herein as a medicament) which comprises the therapeutic agent and one or more pharmaceutically acceptable carriers, excipients, or diluents, according to pharmaceutical practice.

The term “sequential” or “sequentially” refers to the administration of each therapeutic agent of the combination therapy, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

The term “concurrently” refers to the administration of each therapeutic agent in a combination therapy, either alone or in separate medicaments, wherein the second therapeutic agent is administered immediately after the first therapeutic agent, but that the therapeutic agents can be administered in any order. In a preferred embodiment the therapeutic agents are administered concurrently.

The term “simultaneous” refers to the administration of each therapeutic agent of the combination therapy in the same medicament, for example as a fixed dose combination comprising two or more drugs in a single dosage form.

A “dosing regimen” refers to the period of administration of one or more drugs, compounds or compositions, comprising one or more treatment cycles, wherein each treatment cycle may include administration of one or more agents at different times, frequencies or amounts, using the same or different routes of administration. Repetition of the administration or dosing regimens, or adjustment of the administration or dosing regimen may be conducted as necessary to achieve the desired treatment effect.

“BID” or “bid” refers to administration of a drug, compound or composition twice a day.

“QD” or “qd” refers to administration of a drug, compound or composition once a day.

“TID” or “tid” refers to administration of a drug, compound or composition three times a day.

The term “additive” means the result of the combination of two or more drugs, compounds or compositions is no greater than the sum of each drug, compound or composition individually.

The term “synergy” or “synergistic” means that the result of the combination of two or more drugs, compounds or compositions is greater than the sum of each drug, compound or composition individually. Combinations providing a synergistic effect may be referred to as synergistic combinations.

A “synergistic amount” is an amount of the two or more drugs, compounds or compositions in the combination that results in a synergistic effect. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol. Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Ma & Motsinger-Reif, Current Method for Quantifying Drug Synergism, Proteom. Bioinform (2019) 1(2):43-48; Tang et al., What is Synergy? The Saariselkä Agreement Revisited, Front Pharmacol. (2015) Article 181, 6: 1-5.

All references herein to CDK2 inhibitors of Formula (I), or to CDK4 inhibitors of Formula (II) include (to the extent chemically feasible) references to pharmaceutically acceptable salts, solvates, hydrates and complexes thereof, and to solvates, hydrates and complexes of pharmaceutically acceptable salts thereof, and include amorphous and polymorphic forms, stereoisomers, and isotopically labeled versions thereof.

As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein. For example, compounds that are basic in nature may be capable of forming a wide variety of salts with various inorganic and organic acids. The acids that may be used to prepare pharmaceutically acceptable acid addition salts of such basic compounds of those that form non-toxic acid addition salts, i.e., salts containing pharmacologically acceptable anions. Examples of anions suitable for mono- and di-acid addition salts include, but are not limited to, acetate, asparatate, benzenesulfonate, benzoate, besylate, bicarbonate, bisulfate, bitartrate, bromide, calcium edetate, camsylate, carbonate, chloride, citrate, decanoate, edetate, edislyate, estolate, esylate, fumarate, gluceptate, gluconate, glutamate, glycollate, hexanoate, hexylresorcinate, hydrabamine, hydroxynaphthoate, iodide, isethionate, lactate, lactobionate, malate, maleate, mandelate, mesylate, methylsulfate, mucate, napsylate, nitrate, octanoate, oleate, pamoate (embonate), pantothenate, phosphate, polygalacturonate, propionate, salicylate, stearate, subacetate, succinate, sulfate, tannate, tartrate, teoclate, tosylate, triethiodode, and valerate salts. Alternatively, compounds that are acidic in nature may be capable of forming base salts with various pharmacologically acceptable cations which form non-toxic base salts. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. Examples of cations suitable for such salts include alkali metal or alkaline-earth metal salts and other cations, including aluminium, arginine, benzathine, calcium, chloroprocaine, choline, diethanolamine, ethanolamine, ethylenediamine, lysine, magnesium, histidine, lithium, meglumine, potassium, procaine, sodium, triethyamine and zinc. Salts may be prepared by conventional techniques. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002). Methods for making pharmaceutically acceptable salts are known to those of skill in the art.

### Therapeutic Methods, Combinations, Uses

The present disclosure provides methods, combinations, and uses comprising a CDK2 inhibitor, which is a compound of Formula (I):

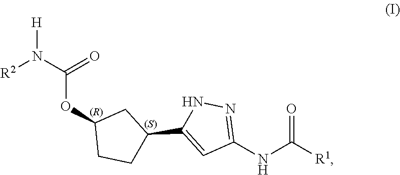

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy.

In each instance recited herein, reference to “a compound of Formula (I)” may be replaced by “a CDK2 inhibitor of Formula (I).”

The present disclosure further provides methods, combinations, and uses comprising a selective CDK4 inhibitor, which is a compound of Formula (II):

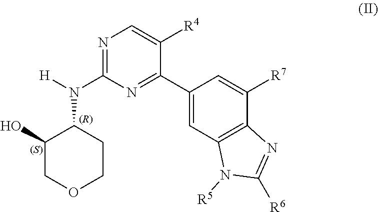

or a pharmaceutically acceptable salt or solvate thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl.

In one aspect, the disclosure provides a method of treating cancer in a subject in need thereof comprising administering to the subject:
(a) an amount of a compound of Formula (I):

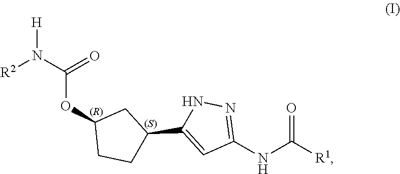

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) an amount of a compound of Formula (II):

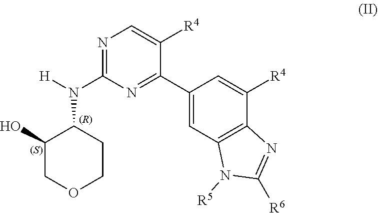

or a pharmaceutically acceptable salt or solvate thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl;
wherein the amounts in (a) and (b) together are effective in treating cancer.

In some embodiments, the method further comprises administering to the subject (c) an amount of an additional anti-cancer agent; wherein the amounts in (a), (b) and (c) together are effective in treating cancer.

In another aspect, the disclosure provides a combination comprising:
(a) a compound of Formula (I):

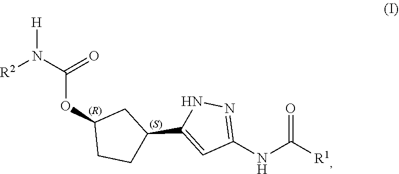

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) a compound of Formula (II):

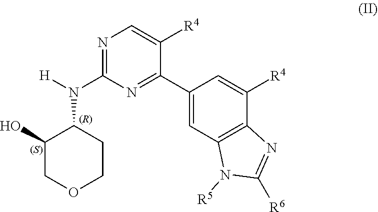

or a pharmaceutically acceptable salt or solvate thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl;
wherein the combination of (a) and (b) is effective in treating cancer.

In some embodiments, the combination further comprises (c) an additional anti-cancer agent; wherein the combination of (a), (b) and (c) is effective in treating cancer.

In another aspect, the disclosure provides a combination for use in treating cancer comprising:
(a) a compound of Formula (I):

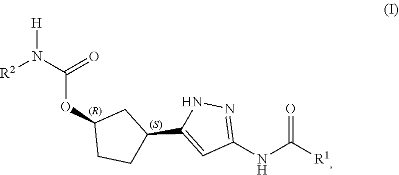

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) a compound of Formula (II):

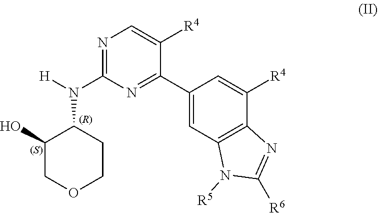

or a pharmaceutically acceptable salt or solvate thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl.

In some embodiments, the combination for use further comprises (c) an additional anti-cancer agent.

In another aspect, the disclosure provides use of a combination comprising:
(a) a compound of Formula (I):

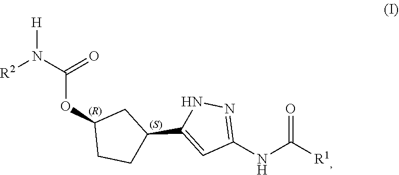

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) a compound of Formula (II):

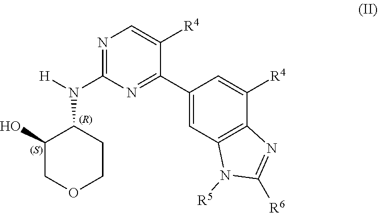

or a pharmaceutically acceptable salt or solvate thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl;
wherein use of the combination of (a) and (b) is effective in treating cancer.

In some embodiments of this aspect, the combination further comprises (c) an additional anti-cancer agent, wherein the use of the combination of (a), (b) and (c) is effective in treating cancer.

In some embodiments of each of the methods, combinations and uses described herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091), having the structure:

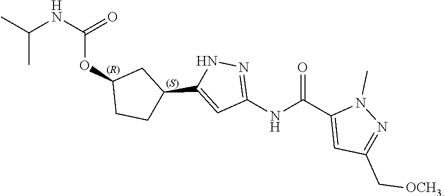

It is understood that the above structural formula of PF-07104091 includes all tautomeric forms which may co-exist and be directly interconverted under the appropriate conditions. For example, in some embodiments, PF-07104091 has a structure of:

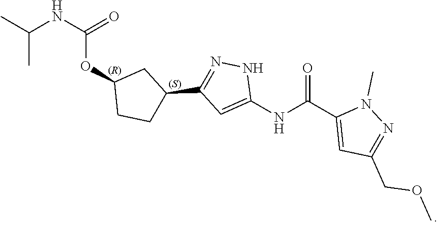

In preferred embodiments of each of the methods, combinations and uses described herein, the compound of Formula (II) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (PF-07220060), having the structure:

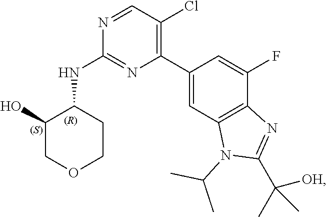

or a pharmaceutically acceptable salt thereof.

In another aspect, the disclosure provides a method of treating cancer in a subject in need thereof comprising administering to the subject:
(a) an amount of PF-07104091 monohydrate; and
(b) an amount of PF-07220060 or a pharmaceutically acceptable salt thereof;
wherein the amounts in (a) and (b) together are effective in treating cancer.

In another aspect, the disclosure provides a method of treating cancer in a subject in need thereof comprising administering to the subject:
(a) an amount of PF-07104091 monohydrate;
(b) an amount of PF-07220060 or a pharmaceutically acceptable salt thereof; and
(c) an amount of an additional anti-cancer agent;
wherein the amounts in (a), (b) and (c) together are effective in treating cancer.

In another aspect, the disclosure provides a method of treating cancer in a subject in need thereof comprising administering to the subject:
(a) an amount of PF-07104091 monohydrate; and
(b) an amount of a selective CDK4 inhibitor;
wherein the amounts in (a) and (b) together are effective in treating cancer.

In another aspect, the disclosure provides a method of treating cancer in a subject in need thereof comprising administering to the subject:
(a) an amount of PF-07104091 monohydrate;
(b) an amount of a selective CDK4 inhibitor; and
(c) an amount of an additional anti-cancer agent;
wherein the amounts in (a), (b) and (c) together are effective in treating cancer.

In another aspect, the disclosure provides a combination comprising:
(a) PF-07104091 monohydrate; and
(b) PF-07220060 or a pharmaceutically acceptable salt thereof;
wherein the combination of (a) and (b) is effective in treating cancer.

In another aspect, the disclosure provides a combination comprising:
(a) PF-07104091 monohydrate;
(b) PF-07220060 or a pharmaceutically acceptable salt thereof; and
(c) an additional anti-cancer agent;
wherein the combination of (a), (b) and (c) is effective in treating cancer.

In another aspect, the disclosure provides a combination comprising:
(a) PF-07104091 monohydrate; and
(b) a selective CDK4 inhibitor;
wherein the combination of (a) and (b) is effective in treating cancer.

In another aspect, the disclosure provides a combination comprising:
(a) PF-07104091 monohydrate;
(b) a selective CDK4 inhibitor; and
(c) an additional anti-cancer agent;
wherein the combination of (a), (b) and (c) is effective in treating cancer.

In another aspect, the disclosure provides a combination for use in treating cancer comprising:
(a) PF-07104091 monohydrate; and
(b) PF-07220060 or a pharmaceutically acceptable salt thereof.

In another aspect, the disclosure provides a combination for use in treating cancer comprising:
(a) PF-07104091 monohydrate;
(b) PF-07220060 or a pharmaceutically acceptable salt thereof; and
(c) an additional anti-cancer agent.

In another aspect, the disclosure provides a combination for use in treating cancer comprising:
(a) PF-07104091 monohydrate; and
(b) a selective CDK4 inhibitor.

In another aspect, the disclosure provides a combination for use in treating cancer comprising:
(a) PF-07104091 monohydrate;
(b) a selective CDK4 inhibitor; and
(c) an additional anti-cancer agent.

In another aspect, the disclosure provides use of a combination comprising:
(a) PF-07104091 monohydrate; and
(b) PF-07220060 or a pharmaceutically acceptable salt thereof;
wherein use of the combination of (a) and (b) is effective in treating cancer.

In another aspect, the disclosure provides use of a combination comprising:
(a) PF-07104091 monohydrate;
(b) PF-07220060 or a pharmaceutically acceptable salt thereof; and
(c) an additional anti-cancer agent;
wherein use of the combination of (a), (b) and (c) is effective in treating cancer.

In another aspect, the disclosure provides use of a combination comprising:
(a) PF-07104091 monohydrate; and
(b) a selective CDK4 inhibitor;
wherein use of the combination of (a) and (b) is effective in treating cancer.

In another aspect, the disclosure provides use of a combination comprising:
(a) PF-07104091 monohydrate;
(b) a selective CDK4 inhibitor; and
(c) an additional anti-cancer agent.
wherein use of the combination of (a), (b) and (c) is effective in treating cancer.

In some embodiments of each of the methods, combinations, and uses described herein, the cancer is selected from the group consisting of breast cancer, prostate cancer, lung cancer (including non-small cell lung cancer, NSCLC, and small cell lung cancer, SCLC), liver cancer (including hepatocellular carcinoma, HCC), kidney cancer (including renal cell carcinoma, RCC), bladder cancer (including urothelial carcinomas, such as upper urinary tract urothelial carcinoma, UUTUC), ovarian cancer (including epithelial ovarian cancer, EOC), peritoneal cancer (including primary peritoneal cancer, PPC), fallopian tube cancer, cervical cancer, uterine cancer (including endometrial cancer), pancreatic cancer, stomach cancer, colorectal cancer, esophageal cancer, head and neck cancer (including squamous cell carcinoma of the head and neck (SCCHN), thyroid cancer, and salivary gland cancer), testicular cancer, adrenal cancer, skin cancer (including basal cell carcinoma and melanoma), brain cancer (including astrocytoma, meningioma, and glioblastoma), sarcoma (including osteosarcoma and liposarcoma), and lymphoma (including mantle cell lymphoma, MCL).

In some such embodiments, the cancer is selected from the group consisting of breast cancer, lung cancer, ovarian cancer, peritoneal cancer, fallopian tube cancer, bladder cancer, uterine cancer, colon cancer, prostate cancer, esophageal cancer, liver cancer, pancreatic cancer and stomach cancer.

In some embodiments of each of the methods, combinations and uses described herein, the cancer is characterized by amplification or overexpression of cyclin E1 (CCNE1) and/or cyclin E2 (CCNE2). In some such embodiments, the cancer is characterized by amplification or overexpression of cyclin E1 (CCNE1).

Examples of cyclin E-dominant cancers include, but are not limited to, ovarian cancer, breast cancer, liver cancer, stomach cancer, esophageal cancer, bladder cancer, uterine cancer, small cell lung cancer (SCLC), non-small cell lung cancer (NSCLC), colon cancer, prostate cancer or pancreatic cancer.

The retinoblastoma susceptibility gene (RB1) was the first tumor suppressor gene to be molecularly defined. The retinoblastoma gene product, RB, is frequently mutated or deleted in retinoblastoma and osteosarcoma, and is mutated or deleted with variable frequency in other tumor types, such as prostate cancer (including neuroendocrine prostate carcinoma), breast cancer (including triple negative breast cancer, TNBC), lung cancer (including small cell lung cancer, SCLC, and non-small cell lung cancer, NSCLC), liver cancer, bladder cancer, ovarian cancer, uterine cancer, cervical cancer, stomach cancer, esophageal cancer, head and neck cancer, glioblastoma, and lymphoma. In human cancers, the function of RB may be disrupted through neutralization by a binding protein, (e.g., the human papilloma virus-E7 protein in cervical carcinoma; Ishiji, T, 2000[0021], J Dermatol., 27: 73-86) or deregulation of pathways ultimately responsible for its phosphorylation.

By “RB pathway” it is meant the entire pathway of molecular signaling that includes retinoblastoma protein (RB), and other protein/protein families in the pathway, including but not limited to CDK, E2f, atypical protein kinase C, and Skp2. Inactivation of the RB pathway often results from perturbation of p161NK4a, Cyclin D1, and CDK4.

The terms “RB+,” “RB plus,” “RB-proficient” or “RB-positive” may be used to describe cells expressing detectable amounts of functional RB protein. RB-positive includes wild-type and non-mutated RB protein. A wild-type RB (RB-WT) is generally understood to mean that form of the RB protein which is normally present in a corresponding population and which has the function which is currently assigned to this protein. RB-positive may be cells which contain a functional RB gene. Cells which are RB-positive may also be cells that can encode a detectable RB protein function.

The terms “RB−,” “RB minus,” “RB-deficient” or “RB-negative” describe several types of cell where the function of RB is disrupted, including cells which produce no detectable amounts of functional RB protein. Cells that are RB-negative may be cells which do not contain a functional RB gene. Cells that are RB-negative may also be cells that can encode an RB protein, but in which the protein does not function properly.

In some embodiments of each of the methods and uses described herein, the cancer is characterized as retinoblastoma wild type (RB-WT). In some embodiments of each of the methods and uses described herein, the cancer is characterized as RB-positive or RB-proficient. Such RB-positive or RB-proficient cancers contain at least some functional retinoblastoma genes. In some embodiments, such RB-WT, RB-positive or RB-proficient cancers are characterized as RB1-WT, RB1-positive or RB1-proficient cancers.

In some embodiments of each of the methods, combinations and uses described herein, the cancer is characterized as RB-negative or RB-deficient. Such RB-negative or RB-deficient cancers may be characterized by loss of function mutations, which may encode missense mutations (i.e., encode the wrong amino acid) or nonsense mutations (i.e., encode a stop codon). Alternatively, such RB-negative cancers may be characterized by deletion of all or part of the retinoblastoma gene. In some embodiments, such RB-negative or RB-deficient cancers are characterized as RB1-negative or RB1-deficient.

In some embodiments of each of the methods, combinations and uses described herein, the cancer is advanced or metastatic cancer.

In some embodiments of each of the methods, combinations and uses described herein, the cancer is refractory, i.e., the cancer does not respond at all to treatment with a therapeutic agent or class (including a standard of care agent or class) or initially responds but starts to grow again in a very short period of time.

In some embodiments of each of the methods, combinations and uses described herein, the cancer is resistant to a therapeutic agent or class (including a standard of care agent or class). In some embodiments of each of the methods, combinations and uses described herein, the cancer is characterized by innate or acquired resistance to a therapeutic agent or class (including a standard of care agent or class).

In some embodiments of each of the methods, combinations and uses described herein, the compound of Formula (I) or a pharmaceutically acceptable salt or solvate thereof, and the compound of Formula (II) or a pharmaceutically acceptable salt or solvate thereof, are administered sequentially, simultaneously, or concurrently. In some embodiments of each of the methods, combinations and uses described herein, PF-07104091 or a pharmaceutically acceptable salt or solvate thereof, and PF-07220060 or a pharmaceutically acceptable salt or solvate thereof are administered sequentially, simultaneously or concurrently. In some embodiments of each of the methods, combinations and uses described herein, PF-07104091 and a selective CDK4 inhibitor are administered sequentially, simultaneously or concurrently.

In preferred embodiments of the methods, combinations and uses described herein, the cancer is breast cancer. In some embodiments of each of the breast cancer subtypes described herein, the breast cancer is advanced or metastatic breast cancer.

In some embodiments, the breast cancer is hormone receptor positive (HR+), i.e., the breast cancer is estrogen receptor positive (ER+) and/or progesterone receptor positive (PR+). In some embodiments, the breast cancer is hormone receptor negative (HR−), i.e., the breast cancer is estrogen receptor negative (ER−) and progesterone receptor negative (PR−).

In some embodiments wherein the cancer is HR+ breast cancer, the methods, combinations and uses described herein further comprise an additional anti-cancer agent, wherein the additional anti-cancer agent is an endocrine therapeutic agent. In some such embodiments, the endocrine therapeutic agent is an aromatase inhibitor, a SERD or a SERM. In preferred embodiments, the endocrine therapeutic agent is letrozole or fulvestrant.

In some embodiments, the breast cancer is human epidermal growth factor receptor 2 negative (HER2−). In some embodiments, the breast cancer is human epidermal growth factor receptor 2 positive (HER2+).

In some embodiments, the breast cancer is associated with the BRCA1 or BRCA2 gene.

In preferred embodiments, the breast cancer is HR+/HER2− breast cancer. In some such embodiments, the HR+/HER2− breast cancer is refractory to treatment with or has progressed on treatment with a CDK4/6 inhibitor. In some such embodiments, the HR+/HER2− breast cancer is resistant to treatment with a CDK4/6 inhibitor, such as palbociclib or a pharmaceutically acceptable salt thereof. In some embodiments, the HR+/HER2− breast cancer is characterized by amplification or overexpression of cyclin E1 (CCNE1) and/or cyclin E2 (CCNE2). In some embodiments, the HR+/HER2− breast cancer is characterized by amplification or overexpression of cyclin E1 (CCNE1). In some embodiments of each of the foregoing, the HR+/HER2− breast cancer is advanced or metastatic HR+/HER2− breast cancer.

In some embodiments, the breast cancer is HR+/HER2+ breast cancer. In other embodiments, the breast cancer is HR−/HER2+ breast cancer. In some embodiments wherein the breast cancer is HER2+, the methods, combinations and uses described herein further comprise an additional anti-cancer agent, wherein the additional anti-cancer agent is a HER2-targeted agent (e.g., trastuzumab emtansine, fam-trastuzumab deruxtecan, pertuzumab, lapatinib, neratinib or tucatinib), or an agent targeting the PI3K/AKT molecular pathway (e.g., ipatasertib).

In other embodiments, the breast cancer is triple negative breast cancer (TNBC), i.e., the breast cancer is ER−, PR− and HER2−. In some embodiments, the TNBC is refractory or resistant to treatment with a CDK4/6 inhibitor, such as palbociclib or a pharmaceutically acceptable salt thereof. In some embodiments, the TNBC is characterized by amplification or overexpression of cyclin E1 (CCNE1) and/or cyclin E2 (CCNE2). In some such embodiments, the TNBC is characterized by amplification or overexpression of cyclin E1 (CCNE1). In some embodiments, the TNBC is advanced or metastatic TNBC.

In some embodiments of each of the foregoing, the breast cancer is refractory to treatment with one or more standard of care agents. In some embodiments of each of the foregoing, the breast cancer is resistant to treatment with one or more standard of care agents.

In some such embodiments, the breast cancer is refractory or resistant to treatment with endocrine therapeutic agents, such as aromatase inhibitors, SERDs, or SERMs. In some embodiments, the breast cancer is refractory or resistant to treatment with, or has progressed on, a CDK4/6 inhibitor. In other embodiments, the breast cancer is refractory or resistant to treatment with, or has progressed on, treatment with antineoplastic chemotherapeutic agents such as platinum agents, taxanes, anthracyclines or anti-metabolites.

In some embodiments of each of the methods, combinations and uses described herein, the cancer is lung cancer. In some such embodiments, the lung cancer is advanced or metastatic lung cancer.

In some such embodiments, the lung cancer is small cell lung cancer (SCLC). In some such embodiments, the SCLC is characterized by loss of retinoblastoma (RB) function. In some such embodiments, the SCLC is advanced or metastatic SCLC. In some such embodiments, the SCLC is advanced or metastatic SCLC characterized by loss of retinoblastoma (RB) function.

In some embodiments of the methods and uses described herein, the cancer is SCLC. In some such embodiments, the SCLC is Rb-negative or Rb-deficient.

In some embodiments of the methods and uses described herein, the cancer is NSCLC. In some such embodiments, the NSCLC is characterized by amplification or overexpression of cyclin E1 (CCNE1) and/or cyclin E2 (CCNE2). In some embodiments, the NSCLC is characterized by amplification or overexpression of cyclin E1 (CCNE1). In some such embodiments, the NSCLC is advanced or metastatic NSCLC. In some such embodiments, the NSCLC is advanced or metastatic NSCLC characterized by amplification or overexpression of cyclin E1 (CCNE1). In other embodiments, the NSCLC is lung squamous cell carcinoma (LUSC) or lung adenocarcinoma (LUAD). In a preferred embodiment, the lung cancer is a LUAD. Single-gene drive oncogene drivers of lung adenocarcinomas include, but are not limited to, EGFR, BRAF, and KRAS. About 25% of lung adenocarcinomas are KRAS driven, which may include KRAS G12C and non-G12C driven segments, such as G12A, G12D, G12V, G13D and L19F driven tumors.

In some embodiments, the cancer is lung cancer, including SCLC or NSCLC, and the methods, combinations and uses described herein further comprise an additional anti-cancer agent.

In some embodiments of each of the methods, combinations and uses described herein, the cancer is ovarian cancer, peritoneal cancer, or fallopian tube cancer (FTC). In some such embodiments, the ovarian cancer is epithelial ovarian cancer (EOC). In some such embodiments, the peritoneal cancer is primary peritoneal carcinomatosis (PPC). In some embodiments, the cancer is epithelial ovarian cancer (EOC), primary peritoneal carcinomatosis (PPC) or fallopian tube cancer (FTC). In some embodiments, the ovarian cancer is persistent, refractory or recurrent ovarian cancer. In some embodiments, the ovarian cancer is platinum resistant ovarian cancer. In some such embodiments, the cancer is advanced or metastatic ovarian cancer. In some such embodiments, the cancer is platinum resistant advanced or metastatic ovarian cancer. In some such embodiments, the cancer is advanced or metastatic EOC, PPC or FTC. In some such embodiments, the cancer is platinum resistant advanced or metastatic EOC, PPC or FTC.

In some embodiments, each of the methods, combinations and uses described herein further comprise an additional anti-cancer agent. In some embodiments the additional anti-cancer agent is a standard of care agent for the type of cancer. In some embodiments, each of the methods, combinations and uses described herein further comprise an additional anti-cancer agent, wherein the compound of Formula (I) or a pharmaceutically acceptable salt or solvate thereof, the compound of Formula (II) or a pharmaceutically acceptable salt thereof, and the additional anti-cancer agent are administered sequentially, simultaneously or concurrently.

In some embodiments of each of the methods, combinations and uses described herein, the cancer is breast cancer (including HR+ or HR+/HER2− breast cancer), and the methods, combinations and uses further comprise an additional anti-cancer agent. In some embodiments, the additional anti-cancer agent is an endocrine therapeutic agent. In some such embodiments, the endocrine therapeutic agent is an aromatase inhibitor, a SERD, or a SERM. In some embodiments, the endocrine therapeutic agent is an aromatase inhibitor. In some such embodiments, the aromatase inhibitor is selected from the group consisting of letrozole, anastrozole, and exemestane. In some preferred embodiments, the aromatase inhibitor is letrozole. In some embodiments, the endocrine therapeutic agent is a SERD. In some such embodiments, the SERD is selected from the group consisting of fulvestrant, elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), and SHR9549 (Jiansu Hengrui Medicine). In some preferred embodiments, the SERD is fulvestrant. In some embodiments, the endocrine therapeutic agent is a SERM. In some such embodiments, the SERM is selected from the group consisting of tamoxifen, raloxifene, toremifene, lasofoxifene, bazedoxifene and afimoxifene. In some such embodiments, the SERM is tamoxifen or raloxifene.

In some embodiments of each of the methods, combinations and uses described herein, the cancer is breast cancer (including HR+ or HR+/HER2− breast cancer), and the methods, combinations and uses further comprise an additional anti-cancer agent, wherein the compound of Formula (I) is PF-07104091, the compound of Formula (II) is PF-07220060 or a pharmaceutically acceptable salt thereof, and the additional anti-cancer agent is an endocrine therapeutic agent, wherein the endocrine therapeutic agent is an aromatase inhibitor, a SERD, or a SERM. In some such embodiments, the endocrine therapeutic agent is letrozole or fulvestrant.

### Pharmaceutical Compositions, Medicaments and Kits

The present disclosure further provides pharmaceutical compositions, medicaments and kits comprising a compound of Formula (I):

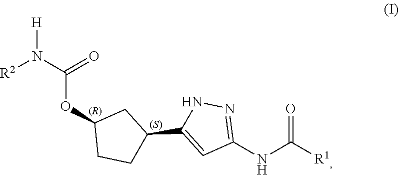

or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy.

In another aspect, the disclosure provides a pharmaceutical composition comprising a compound of Formula (I) or a pharmaceutically acceptable salt or solvate thereof, a selective CDK4 inhibitor or a pharmaceutically acceptable salt thereof, and a pharmaceutically acceptable carrier or excipient. In some embodiments of this aspect, the pharmaceutical composition further comprises an additional anti-cancer agent (e.g., an endocrine therapeutic agent).

In another aspect, the disclosure provides a first pharmaceutical composition comprising a compound of Formula (I) or a pharmaceutically acceptable salt or solvate thereof, and a pharmaceutically acceptable carrier or excipient, and a second pharmaceutical composition comprising a compound of Formula (II) or a pharmaceutically acceptable salt thereof, and a pharmaceutically acceptable carrier or excipient, wherein the first and second pharmaceutical compositions are administered sequentially, simultaneously or concurrently. Some embodiments of this aspect further comprise a third pharmaceutical composition comprising an additional anti-cancer agent (e.g., an endocrine therapeutic agent) and a pharmaceutically acceptable carrier or excipient, wherein the first, second and third pharmaceutical compositions are administered sequentially, simultaneously, or concurrently.

In another aspect, the disclosure provides a combination comprising a compound of Formula (I) or a pharmaceutically acceptable salt or solvate thereof, and a selective CDK4 inhibitor or a pharmaceutically acceptable salt thereof, for use in the manufacture of a medicament for treating cancer in a subject.

In another aspect, the disclosure provides use of a combination comprising PF-07104091 or a pharmaceutically acceptable solvate thereof, and PF-07220060 or a pharmaceutically acceptable salt thereof, in the manufacture of a medicament for treating cancer in a subject. In some embodiments of these aspects, the combination further comprises an additional anti-cancer agent (e.g., an endocrine therapeutic agent) for use in the manufacture of a medicament.

In another aspect, the disclosure provides a compound of Formula (I) or a pharmaceutically acceptable salt or solvate thereof for use in the manufacture of a medicament for treating cancer, wherein the medicament is adapted for use in combination with a compound of Formula (II) or a pharmaceutically acceptable salt thereof. In another aspect, the disclosure provides a compound of Formula (I) or a pharmaceutically acceptable salt or solvate thereof for use in the manufacture of a medicament for treating cancer, wherein the medicament is adapted for use in combination with a compound of Formula (II) or a pharmaceutically acceptable salt thereof, and an additional anti-cancer agent, e.g., an endocrine therapeutic agent.

In preferred embodiments of each of the pharmaceutical compositions, kits and medicaments described herein, the compound of Formula (I) is PF-07104091 or a monohydrate thereof. In preferred embodiments of each of the pharmaceutical compositions, kits and medicaments described herein, the compound of Formula (II) is PF-07220060 or a pharmaceutically acceptable salt thereof.

In particularly preferred embodiments of each of pharmaceutical compositions, kits and medicaments described herein, the compound of Formula (I) is PF-07104091 monohydrate and the compound of Formula (II) is PF-07220060.

In another aspect, the disclosure provides a kit comprising a first container, a second container and a package insert, wherein the first container comprises at least one dose of a compound of Formula (I) or a pharmaceutically acceptable salt or solvate thereof; the second container comprises at least one dose of a compound of Formula (II) or a pharmaceutically acceptable salt thereof; and the package insert comprises instructions for treating cancer in a subject using the medicaments. In some embodiments, the kit further comprises a third container, wherein the third container comprises at least one dose of an additional anti-cancer agent (e.g., an endocrine therapeutic agent); and the package insert comprises instructions for treating cancer in a subject using the medicaments.

In embodiments of the pharmaceutical compositions, medicaments, and kits comprising an additional anti-cancer agent, the additional anti-cancer agent is an endocrine therapeutic agent, such as an aromatase inhibitor, a SERD, or a SERM. In some such embodiments, the endocrine therapeutic agent is letrozole or fulvestrant.

The pharmaceutical compositions, medicaments and kits described herein may be useful for treating the cancers described above with respect to the methods, combinations and uses disclosed herein. In some embodiments, the pharmaceutical compositions, medicaments and kits may be useful for treating cancer selected from the group consisting of breast cancer (including HR+/HER2−, HR+/HER2+, HR−/HER2+ or TNBC), lung cancer (including SCLC or NSCLC), ovarian cancer (including EOC), peritoneal cancer (including PPC), fallopian tube cancer (including FTC), bladder cancer, colon cancer, uterine cancer, prostate cancer, esophageal cancer, liver cancer, pancreatic cancer and stomach cancer.

### Dosage Forms and Regimens

Each therapeutic agent included in the combinations, dosage regimens, methods and uses herein may be administered either alone, or as a medicament (also referred to herein as a pharmaceutical composition) which comprises the therapeutic agent and one or more pharmaceutically acceptable carriers, excipients, or diluents, according to pharmaceutical practice.

As used herein, the terms “combination” or “combination therapy” refer to the administration of two or more therapeutic agents of the combination therapy, either as a compound or in the form of a pharmaceutical composition. The combination therapy may be administered sequentially, concurrently or simultaneously.

In certain embodiments, the effective amount of PF-07104091 when used in combination with PF-07220060 on a daily basis (i.e., total daily dose) is from about 100 mg to about 500 mg per day, from about 100 mg to about 450 mg per day, from about 100 mg to about 400 mg per day, from about 100 mg to about 375 mg per day, from about 100 mg to about 350 mg per day, from about 100 mg to about 325 mg per day, from about 100 mg to about 300 mg per day, from about 100 mg to about 275 mg per day, from about 100 mg to about 250 mg per day, from about 100 mg to about 225 mg per day, from about 100 mg to about 200 mg per day, from about 100 mg to about 175 mg per day, from about 100 mg to about 150 mg per day, from about 150 mg to about 500 mg per day, from about 150 mg to about 450 mg per day, from about 150 mg to about 400 mg per day, from about 150 mg to about 375 mg per day, from about 150 mg to about 350 mg per day, from about 150 mg to about 325 mg per day, from about 150 mg to about 300 mg per day, from about 150 mg to about 275 mg per day, from about 150 mg to about 250 mg per day, from about 150 mg to about 225 mg per day, from about 150 mg to about 200 mg per day, from about 200 mg to about 500 mg per day, from about 200 mg to about 450 mg per day, from about 200 mg to about 400 mg per day, from about 200 mg to about 375 mg per day, from about 200 mg to about 350 mg per day, from about 200 mg to about 325 mg per day, from about 200 mg to about 300 mg per day, from about 200 mg to about 275 mg per day, or from about 200 mg to about 250 mg per day.

In certain embodiments, the effective amount of PF-07104091 when administered QD in combination with PF-07220060 is from about 100 mg to about 500 mg QD, from about 100 mg to about 450 mg QD, from about 100 mg to about 400 mg QD, from about 100 mg to about 375 mg QD, from about 100 mg to about 350 mg QD, from about 100 mg to about 325 mg QD, from about 100 mg to about 300 mg QD, from about 100 mg to about 275 mg QD, from about 100 mg to about 250 mg QD, from about 100 mg to about 225 mg QD, from about 100 mg to about 200 mg QD, from about 100 mg to about 175 mg QD, from about 100 mg to about 150 mg QD, from about 150 mg to about 500 mg QD, from about 150 mg to about 450 mg QD, from about 150 mg to about 400 mg QD, from about 150 mg to about 375 mg QD, from about 150 mg to about 350 mg QD, from about 150 mg to about 325 mg QD, from about 150 mg to about 300 mg QD, from about 150 mg to about 275 mg QD, from about 150 mg to about 250 mg QD, from about 150 mg to about 225 mg QD, from about 150 mg to about 200 mg QD, from about 200 mg to about 500 mg QD, from about 200 mg to about 450 mg QD, from about 200 mg to about 400 mg QD, from about 200 mg to about 375 mg QD, from about 200 mg to about 350 mg QD, from about 200 mg to about 325 mg QD, from about 200 mg to about 300 mg QD, from about 200 mg to about 275 mg QD, from about 200 mg to about 250 mg QD.

In preferred embodiments, the effective amount of PF-07104091 when administered BID in combination with PF-07220060 is from about 50 mg to about 250 mg BID, from about 50 mg to about 225 mg BID, from about 50 mg to about 200 mg BID, from about 50 mg to about 175 mg BID, from about 50 mg to about 150 mg BID, from about 50 mg to about 125 mg BID, from about 50 mg to about 100 mg BID, from about 75 mg to about 250 mg BID, from about 75 mg to about 225 mg BID, from about 75 mg to about 200 mg BID, from about 75 mg to about 175 mg BID, from about 75 mg to about 150 mg BID, from about 75 mg to about 125 mg BID, from about 75 mg to about 100 mg BID, from about 100 mg to about 250 mg BID, from about 100 mg to about 225 mg BID, from about 100 mg to about 200 mg BID, from about 100 mg to about 175 mg BID, from about 100 mg to about 150 mg BID, OR from about 100 mg to about 125 mg BID,

In some embodiments, PF-07104091 when used in combination with PF-07220060 is administered in a dose of about 100 mg per day, about 150 mg per day, about 200 mg per day, about 250 mg per day, about 300 mg per day, about 350 mg per day, or about 400 mg per day

In some embodiments, PF-07104091 when used in combination with PF-07220060 is administered in a QD dose of about 100 mg QD, about 150 mg QD, about 200 mg QD, about 250 mg QD, about 300 mg QD, about 350 mg QD, or about 400 mg QD.

In some embodiments, PF-07104091 when used in combination with PF-07220060 is administered in a BID dose of about 50 mg BID, about 75 mg BID, about 100 mg BID, about 125 mg BID, about 150 mg BID, about 175 mg BID, or about 200 mg BID.

In preferred embodiments, PF-07104091 is administered on a twice a day (BID) dosing schedule. In preferred embodiments, the effective amount of PF-07104091 when used in combination with PF-07220060 is about 75 mg BID, about 100 mg BID, about 125 mg BID, or about 150 mg BID.

In certain embodiments, the therapeutically effective amount of PF-07220060 when used in combination with PF-07104091 is from about 200 mg to about 1000 mg per day (i.e., total daily dose), for example, from about 200 mg to about 500 mg, from about 200 mg to about 450 mg, from about 200 mg to about 400 mg, from about 200 mg to about 350 mg, from about 200 mg to about 300 mg, from about 400 mg to about 950 mg, from about 400 mg to about 900 mg, from about 400 mg to about 850 mg, from about 400 mg to about 800 mg, from about 500 mg to about 950 mg, from about 500 mg to about 900 mg, from about 500 mg to about 850 mg, from about 500 mg to about 800 mg, from about 600 mg to about 950 mg, from about 600 mg to about 900 mg, from about 600 mg to about 850 mg, or from about 600 mg to about 800 mg per day.

In certain embodiments, PF-07220060 when used in combination with PF-07104091 is administered in doses of from about 200 mg to about 1000 mg once a day (QD), for example, from about 200 mg to about 500 mg QD, from about 200 mg to about 450 mg QD, from about 200 mg to about 400 mg QD, from about 200 mg to about 350 mg QD, from about 200 mg to about 300 mg QD, from about 400 mg to about 950 mg QD, from about 400 mg to about 900 mg QD, from about 400 mg to about 850 mg QD, from about 400 mg to about 800 mg QD, from about 500 mg to about 950 mg QD, from about 500 mg to about 900 mg QD, from about 500 mg to about 850 mg QD, from about 500 mg to about 800 mg QD, from about 600 mg to about 950 mg QD, from about 600 mg to about 900 mg QD, from about 600 mg to about 850 mg QD, or from about 600 mg to about 800 mg QD.

In certain embodiments, the therapeutically effective amount of PF-07220060 when used in combination with PF-07104091 is from about 100 mg to about 500 mg twice a day (BID), for example, from about 200 mg to about 500 mg BID, from about 250 mg to about 500 mg BID, from about 300 mg to about 500 mg BID, from about 350 mg to about 500 mg BID, from about 400 mg to about 500 mg BID, from about 200 mg to about 450 mg BID, from about 250 mg to about 450 mg BID, from about 300 mg to about 450 mg BID, from about 350 mg to about 450 mg BID, from about 400 mg to about 450 mg BID, from about 200 mg to about 400 mg BID, from about 250 mg to about 400 mg BID, from about 300 mg to about 400 mg BID, or from about 350 mg to about 400 mg BID.

In some embodiments, the subject is administered PF-07220060 when used in combination with PF-07104091 at a dose of from about 200 mg to about 1000 mg per day, for example, in daily doses of about 200 mg, about 300 mg, about 400 mg, about 500 mg, about 600 mg, about 700 mg, about 800 mg, about 900 mg, or about 1000 mg.

In some embodiments, PF-07220060 is administered in doses of about 200 mg QD, about 300 mg QD, about 400 mg QD, about 500 mg QD, about 600 mg QD, about 700 mg QD, about 800 mg QD, about 900 mg QD, or about 1000 mg QD.

In preferred embodiments, PF-07220060 when used in combination with PF-07104091 is administered in a dose of about 100 mg BID, about 200 mg BID, about 300 mg BID, about 400 mg BID, or about 500 mg BID.

In some embodiments, the subject is administered PF-07104091 and PF-07220060 at a dose of any of the effective amounts disclosed herein.

In some embodiments, the subject is administered about 75 mg BID PF-07104091 and about 100 mg BID PF-07220060. In some embodiments, the subject is administered about 150 mg BID PF-07104091 and about 100 mg BID PF-07220060. In some embodiments, the subject is administered about 225 mg BID PF-07104091 and about 100 mg BID PF-07220060. In some embodiments, the subject is administered about 75 mg BID PF-07104091 and 200 mg BID PF-07220060. In some embodiments, the subject is administered about 150 mg BID PF-07104091 and about 200 mg BID PF-07220060. In some embodiments, the subject is administered about 225 mg BID PF-07104091 and about PF-07220060. In some embodiments, the subject is administered about 75 mg BID PF-07104091 and about 300 mg BID PF-07220060. In some embodiments, the subject is administered about 150 mg BID PF-07104091 and about 300 mg BID PF-07220060. In some embodiments, the subject is administered about 225 mg BID PF-07104091 and about 300 mg BID PF-07220060.

The amounts of PF-07104091 and PF-07220060 administered may be increased or decreased based on the weight, age, health, sex, or medical condition of the subject. One of skill in the art would be able to determine the proper dose for a subject based on this disclosure.

When administering a combination therapy comprising PF-07104091 and PF-07220060, the agents may be administered on the same treatment cycle or using different treatment cycles.

PF-07104091 and PF-07220060 may be administered in treatment cycles with or without rest periods in between the treatment cycles. A treatment cycle may have a duration of about 7 days, about 14 days, about 21 days, about 28 days, about 35 days and so on, or any days in between. A rest period can be one day or a few days (e.g., 1 day, 2 days, 3 days, 4 days, 5 days, 6 days, and so on), one week, several weeks (e.g., 2 weeks, 3 weeks and so on), or any days in between (e.g., 1 week and 3 days).

In some embodiments, PF-07104091 is administered continuously without a rest period between treatment cycles (i.e., continuous treatment until termination). In some embodiments, PF-07104091 is administered for a treatment cycle (e.g., about 28 days) with or without a rest period. In some embodiments, PF-07104091 is administered for about 28 days with a rest period of about one week. PF-07104091 may be administered for at least about 7 days, about 14 days, about 21 days, about 28 days, about 2 months, about 3 months, about 12 months, about 24 months, and more. In a preferred embodiment, PF-07104091 is administered continuously on a 28 day treatment cycle, without a rest period.

In some embodiments, PF-07220060 is administered continuously without a rest period between treatment cycles (i.e., continuous treatment until termination). In some embodiments, PF-07220060 is administered for a treatment cycle (e.g., about 28 days) with or without a rest period. In some embodiments, PF-07220060 is administered for about 28 days with a rest period of about one week. 07220060 may be administered for at least about 7 days, about 14 days, about 21 days, about 28 days, about 2 months, about 3 months, about 12 months, about 24 months, and more. In a preferred embodiment, 07220060 is administered continuously on a 28 day treatment cycle, without a rest period.

The pharmaceutical compositions may be administered with or without food.

Pharmaceutical compositions may be administered by one or more routes as considered appropriate by a skilled person in the art and depending on the dosage form. The pharmaceutical compositions may be administered with or without food. Formulation of drugs is discussed in Remington's Pharmaceutical Sciences, 18th Ed., (1995) Mack Publishing Co., Easton, Pa. Other examples of drug formulations can be found in Liberman, H. A. and Lachman, L., Eds., Pharmaceutical Dosage Forms, Marcel Decker, Vol 3, 2nd Ed., New York, N.Y. Where the compound is administered orally, it may be formulated as a pill, capsule, tablet, etc. with a pharmaceutically acceptable carrier, glidant, or excipient.

The pharmaceutical compositions may be in one or more dosage forms (e.g., capsule, tablet, powder or liquid).

In some embodiments of each of the methods, combinations and uses herein, the combination therapy is administered to a subject who is previously untreated, i.e., is treatment naïve.

In some embodiments of each of the methods, combinations and uses herein, the combination therapy is administered to a subject who has failed to achieve a sustained response after a prior therapy with a biotherapeutic or chemotherapeutic agent, i.e., is treatment experienced.

In some embodiments of each of the methods, combinations and uses herein, the combination therapy may be administered to a subject who has been previously treated with chemotherapy, radiotherapy, and/or surgical resection.

In certain embodiments of each of the methods, combinations and uses herein, the combination therapy, the subject has been previously treated with a CDK4/6 inhibitor. In some embodiments, the CDK4/6 inhibitor is palbociclib.

In some embodiments of each of the methods, combinations and uses herein, the invention relates to neoadjuvant therapy, adjuvant therapy, first-line therapy, second-line therapy, or third-line or later therapy, in each case for treating cancer as further described herein. In each of the foregoing embodiments, the cancer may be localized, advanced or metastatic, and the intervention may occur at point along the disease continuum (i.e., at any stage of the cancer).

Dosage regimens may be adjusted to provide the optimum desired response. For example, a therapeutic agent of the combination therapy disclosed herein may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms may be dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

In some embodiments, the endocrine therapeutic agent is letrozole, which may be administered orally at a dose of 2.5 mg daily. In some embodiments, the endocrine therapeutic agent is fulvestrant, which may be administered intramuscularly in one or two injections at a dose of 250 mg or 500 mg, respectively, on Days 1, 15, 29, of the first month and then once monthly thereafter.

In some embodiments, the compound of Formula (I) and the compound of Formula (II) are administered on an intermittent dosing schedule. In other embodiments, the compound of Formula (I) and the compound of Formula (II) are administered on a continuous dosing schedule.

In still other embodiments, one of the compound of Formula (I) and the compound of Formula (II) is administered on an intermittent dosing schedule (e.g., a 2/1-week or 3/1-week schedule) and the other is administered on a continuous dosing schedule. In some such embodiments, the compound of Formula (I) is administered on an intermittent dosing schedule and the compound of Formula (II) is administered on a continuous dosing schedule. In other such embodiments, the compound of Formula (I) is administered on a continuous dosing schedule and the compound of Formula (II) is administered on an intermittent dosing schedule.

In some embodiments disclosed herein, the compound of Formula (I) and the compound of Formula (II) are dosed in amounts which together are effective in treating the cancer.

In some embodiments disclosed herein, the compound of Formula (I) and the compound of Formula (II) are dosed in amounts which together are synergistic.

In some embodiments disclosed herein, the compound of Formula (I) and the compound of Formula (II) are dosed in amounts which together are additive.

In preferred embodiments of each of the foregoing, the compound of Formula (I) is PF-07104091 or a monohydrate thereof, and the compound of Formula (II) is PF-07220060 or a pharmaceutically acceptable salt thereof. In particularly preferred embodiments of each of the foregoing, the compound of Formula (I) is PF-07104091 monohydrate and the compound of Formula (II) is PF-07220060.

### Pharmaceutical Compositions and Routes of Administration

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients.

As used herein, a “pharmaceutically acceptable carrier” refers to a carrier or diluent that does not cause significant irritation to an organism and does not abrogate the biological activity and properties of the active compound or therapeutic agent.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, colorants and, optionally, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, solution or suspension, for parenteral injection as a sterile solution, suspension or emulsion, for topical administration as an ointment or cream, or for rectal administration as a suppository.

Exemplary parenteral administration forms include solutions or suspensions of an active compound in a sterile aqueous solution, for example, aqueous propylene glycol or dextrose solutions. Such dosage forms may be suitably buffered, if desired.

The pharmaceutical composition may be in unit dosage forms suitable for single administration of precise amounts.

Pharmaceutical compositions suitable for the delivery of the therapeutic agents of the combination therapies disclosed herein, and methods for their preparation will be readily apparent to those skilled in the art. Such compositions and methods for their preparation may be found, for example, in ‘Remington's Pharmaceutical Sciences’, 19th Edition (Mack Publishing Company, 1995), the disclosure of which is incorporated herein by reference in its entirety.

Therapeutic agents of the combination therapies disclosed herein may be administered orally. Oral administration may involve swallowing, so that the therapeutic agent enters the gastrointestinal tract, or buccal or sublingual administration may be employed by which the therapeutic agent enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups, and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Therapeutic agents of the combination therapies disclosed herein may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the therapeutic agent may make up from 1 wt % to 80 wt % of the dosage form, more typically from 5 wt % to 60 wt % of the dosage form. In addition to the active agent, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch, and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives, and taste-masking agents.

Exemplary tablets may contain up to about 80 wt % active agent, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al., Pharmaceutical Technology On-line, 25(2), 1-14 (2001). The use of chewing gum to achieve controlled release is described in WO 2000/035298. The disclosures of these references are incorporated herein by reference in their entireties.

The kits described herein may be particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like.

### Additional Anti-Cancer Agents

The methods, combinations and uses disclosed herein may additionally comprise one or more additional anti-cancer agents, such as the anti-angiogenesis agents, signal transduction inhibitors or antineoplastic agents described below, wherein the amounts are together effective in treating cancer. In some embodiments, the methods, combinations and uses disclosed herein the additional anti-cancer agents may comprise a palliative care agent. Additional anti-cancer agents may include small molecules therapeutics and pharmaceutically acceptable salts or solvates thereof, therapeutic antibodies, antibody-drug conjugates (ADCs), hetero-bifunctional protein degraders (e.g., proteolysis targeting chimeras or PROTACs), or antisense molecules.

In some embodiments, the methods, combinations and uses disclosed herein further comprise one or more additional anti-cancer agents selected from the following:

Anti-angiogenesis agents include, for example, VEGF inhibitors, VEGFR inhibitors, TIE-2 inhibitors, PDGFR inhibitors, angiopoetin inhibitors, PKCP inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors.

Signal transduction inhibitors include, for example, kinase inhibitors (e.g., inhibitors of tyrosine kinases, serine/threonine kinases or cyclin dependent kinases), proteasome inhibitors, PI3K/AKT/mTOR pathway inhibitors, Phosphoinositide 3-kinase (PI3K) inhibitors, isocitrate dehydrogenase 1 and 2 (IDH1 and IDH2) inhibitors, B-cell lymphoma 2 (BCL2) inhibitors, neurotrophin receptor kinase (NTRK) inhibitors, Rearranged during Transfection (RET) inhibitors, Notch inhibitors, PARP inhibitors, Hedgehog pathway inhibitors, and selective inhibitors of nuclear export (SINE).

Examples of signal transduction inhibitors inhibitors include, but are not limited to: acalabrutinib, afatinib, alectinib, alpelisib, axitinib, binimetinib, bortezomib, bosutinib, brigatinib, cabozantinib, carfilzomib, ceritinib, cobimetinib, copanlisib, crizotinib, dabrafenib, dacomitinib, dasatinib, duvelisib, enasidenib, encorafenib, entrectinib, erlotinib, gefitinib, gilteritinib, glasdegib, ibrutinib, idelalisib, imatinib, ipatasertib, ivosidenib, ixazomib, lapatinib, larotrectinib, lenvatinib, lorlatinib, midostaurin, neratinib, nilotinib, niraparib, olaparib, osimertinib, pazopanib, ponatinib, regorafenib, rucaparib, ruxolitinib, sonidegib, sorafenib, sunitinib, talazoparib, trametinib, vandetanib, vemurafenib, venetoclax, and vismodegib, or pharmaceutically acceptable salts and solvates thereof.

Antineoplastic agents include, for example, alkylating agents, platinum coordination complexes, cytotoxic antibiotics, antimetabolies, biologic response modifiers, histone deacetylate (HDAC) inhibitors, hormonal agents, monoclonal antibodies, growth factor inhibitors, taxanes, topoisomerase inhibitors, Vinca alkaloids and miscellaneous agents.

Alkylating agents include: altretamine, bendamustine, busulfan, carmustine, chlorambucil, cyclophosphamide, dacarbazine, ifosfamide, lomustine, mechlorethamine, melphalan, procarbazine, streptozocin, temozolomide, thiotepa, and trabectedin.

Platinum coordination complexes include: carboplatin, cisplatin, and oxaliplatin.

Cytotoxic antibiotics include: bleomycin, dactinomycin, daunorubicin, doxorubicin, epirubicin, idarubicin, mitomycin, mitoxantrone, plicamycin, and valrubicin.

Antimetabolites include: antifolates, such as methotrexate, pemetrexed, pralatrexate, and trimetrexate; purine analogues, such as azathioprine, cladribine, fludarabine, mercaptopurine, and thioguanine; and pyrimidine analogues such as azacitidine, capecitabine, cytarabine, decitabine, floxuridine, fluorouracil, gemcitabine, and trifluridine/tipracil.

Biologic response modifiers include: aldesleukin (IL-2), denileukin diftitox, and interferon gamma.

Histone deacetylase inhibitors include belinostat, panobinostat, romidepsin, and vorinostat.

Endocrine therapeutic agents (i.e., hormonal therapy agents) include antiandrogens, antiestrogens, gonadotropin releasing hormone (GnRH) analogues and peptide hormones. Examples of antiestrogens include: aromatase inhibitors, such as letrozole, anastrozole, and exemestane; SERDs, such as fulvestrant, elacestrant (RAD-1901, Radius Health/Menarini), amcenestrant (SAR439859, Sanofi), giredestrant (GDC9545, Roche), RG6171 (Roche), camizestrant (AZD9833, AstraZeneca), AZD9496 (AstraZeneca), rintodestrant (G1 Therapeutics), ZN-c5 (Zentalis), LSZ102 (Novartis), D-0502 (Inventisbio), LY3484356 (Eli Lilly), SHR9549 (Jiansu Hengrui Medicine); and SERMs, such as tamoxifen, raloxifene, toremifene, lasofoxifene, bazedoxifene, afimoxifene. Examples of GnRH analogues include: degarelix, goserelin, histrelin, leuprolide, and triptorelin. Examples of peptide hormones include: lanreotide, octreotide, and pasireotide. Examples of antiandrogens include: abiraterone, apalutamide, bicalutamide, cyproterone, enzalutamide, flutamide, and nilutamide, and pharmaceutically acceptable salts and solvates thereof.

Monoclonal antibodies include: alemtuzumab, atezolizumab, avelumab, bevacizumab, blinatumomab, brentuximab, cemiplimab, cetuximab, daratumumab, dinutuximab, durvalumab, elotuzumab, gemtuzumab, inotuzumab ozogamicin, ipilimumab, mogamulizumab, moxetumomab pasudotox, necitumumab, nivolumab, ofatumumab, olaratumab, panitumumab, pembrolizumab, pertuzumab, ramucirumab, rituximab, tositumomab, and trastuzumab.

Taxanes include: cabazitaxel, docetaxel, paclitaxel and paclitaxel albumin-stabilized nanoparticle formulation.

Topoisomerase inhibitors include: etoposide, irinotecan, teniposide, and topotecan.

Vinca alkaloids include: vinblastine, vincristine, and vinorelbine, and pharmaceutically acceptable salts thereof.

Miscellaneous antineoplastic agents include: asparaginase (pegaspargase), bexarotene, eribulin, everolimus, hydroxyurea, ixabepilone, lenalidomide, mitotane, omacetaxine, pomalidomide, tagraxofusp, telotristat, temsirolimus, thalidomide, and venetoclax.

In some embodiments, the additional anti-cancer agent is selected from the group consisting of: abiraterone acetate; acalabrutinib; ado-trastuzumab emtansine; afatinib dimaleate; afimoxifene; aldesleukin; alectinib; alemtuzumab; alpelisib; amifostine; anastrozole; apalutamide; aprepitant; arsenic trioxide; asparaginase Erwinia chrysanthemi; atezolizumab; avapritinib; avelumab; axicabtagene ciloleucel; axitinib; azacitidine; AZD9833 (AstraZeneca); AZD9496 (AstraZeneca); bazedoxifene; belinostat; bendamustine hydrochloride; bevacizumab; bexarotene; bicalutamide; binimetinib; bleomycin sulfate; blinatumomab; bortezomib; bosutinib; brentuximab vedotin; brigatinib; cabazitaxel; cabozantinib-s-malate; calaspargase pegol-mknl; capecitabine; caplacizumab-yhdp; capmatinib hydrochloride; carboplatin; carfilzomib; carmustine; cemiplimab-rwlc; ceritinib; cetuximab; chlorambucil; cisplatin; cladribine; clofarabine; cobimetinib; copanlisib hydrochloride; crizotinib; cyclophosphamide; cytarabine; D-0502 (Inventisbio); dabrafenib mesylate; dacarbazine; dacomitinib; dactinomycin; daratumumab; daratumumab and hyaluronidase-fihj; darbepoetin alfa; darolutamide; dasatinib; daunorubicin hydrochloride; decitabine; defibrotide sodium; degarelix; denileukin diftitox; denosumab; dexamethasone; dexrazoxane hydrochloride; dinutuximab; docetaxel; doxorubicin hydrochloride; durvalumab; duvelisib; elacestrant; elotuzumab; eltrombopag olamine; emapalumab-lzsg; enasidenib mesylate; encorafenib; enfortumab vedotin-ejfv; entrectinib; enzalutamide; epirubicin hydrochloride; epoetin alfa; erdafitinib; eribulin mesylate; erlotinib hydrochloride; etoposide; etoposide phosphate; everolimus; exemestane; fam-trastuzumab deruxtecan-nxki; fedratinib hydrochloride; filgrastim; fludarabine phosphate; fluorouracil; flutamide; fostamatinib disodium; fulvestrant; gefitinib; gemcitabine hydrochloride; gemtuzumab ozogamicin; gilteritinib fumarate; glasdegib maleate; glucarpidase; goserelin acetate; granisetron; granisetron hydrochloride; hydroxyurea; ibritumomab tiuxetan; ibrutinib; idarubicin hydrochloride; idelalisib; ifosfamide; imatinib mesylate; imiquimod; inotuzumab ozogamicin; interferon alfa-2b recombinant; iobenguane 1-131; ipatasertib; ipilimumab; irinotecan hydrochloride; isatuximab-irfc; ivosidenib; ixabepilone; ixazomib citrate; lanreotide acetate; lapatinib ditosylate; larotrectinib sulfate; lasofoxifene; lenalidomide; lenvatinib mesylate; letrozole; leucovorin calcium; leuprolide acetate; lomustine; lorlatinib; LSZ102 (Novartis); lurbinectedin; LY3484356 (Lilly); megestrol acetate; melphalan; melphalan hydrochloride; mercaptopurine; methotrexate; midostaurin; mitomycin; mitoxantrone hydrochloride; mogamulizumab-kpkc; moxetumomab pasudotox-tdfk; necitumumab; nelarabine; neratinib maleate; nilotinib; nilutamide; niraparib tosylate monohydrate; nivolumab; obinutuzumab; ofatumumab; olaparib; omacetaxine mepesuccinate; ondansetron hydrochloride; osimertinib mesylate; oxaliplatin; paclitaxel; paclitaxel albumin-stabilized nanoparticle formulation; palifermin; palonosetron hydrochloride; pamidronate disodium; panitumumab; panobinostat; pazopanib hydrochloride; pegaspargase; pegfilgrastim; peginterferon alfa-2b; pembrolizumab; pemetrexed disodium; pemigatinib; pertuzumab; pexidartinib hydrochloride; plerixafor; polatuzumab vedotin-piiq; pomalidomide; ponatinib hydrochloride; pralatrexate; prednisone; procarbazine hydrochloride; propranolol hydrochloride; radium 223 dichloride; raloxifene hydrochloride; ramucirumab; rasburicase; ravulizumab-cwvz; recombinant interferon alfa-2b; regorafenib; RG6171 (Roche); rintodestrant; ripretinib; rituximab; rolapitant hydrochloride; romidepsin; romiplostim; rucaparib camsylate; ruxolitinib phosphate; sacituzumab govitecan-hziy; SAR439859 (Sanofi); selinexor; selpercatinib; selumetinib sulfate; SHR9549 (Jiansu Hengrui Medicine); siltuximab; sipuleucel-t; sonidegib; sorafenib tosylate; tagraxofusp-erzs; talazoparib tosylate; talimogene laherparepvec; tamoxifen citrate; tazemetostat hydrobromide; temozolomide; temsirolimus; thalidomide; thioguanine; thiotepa; tisagenlecleucel; tocilizumab; topotecan hydrochloride; toremifene; trabectedin; trametinib; trastuzumab; trastuzumab and hyaluronidase-oysk; trifluridine and tipiracil hydrochloride; tucatinib; uridine triacetate; valrubicin; vandetanib; vemurafenib; venetoclax; vinblastine sulfate; vincristine sulfate; vinorelbine tartrate; vismodegib; vorinostat; zanubrutinib; ziv-aflibercept; ZN-c5 (Zentalis); and zoledronic acid; or free base, pharmaceutically acceptable salt, or solvate forms of the foregoing; or combinations thereof.

These and other aspects will be apparent from the teachings contained herein.

PF-07104091 may be prepared as described in U.S. Pat. No. 11,014,911.

PF-07220060 may be prepared as described in U.S. Pat. No. 10,766,884.

### EXAMPLES

The following examples are merely illustrative of the disclosure and should not be considered limiting the scope of the invention, as these examples and other equivalents thereof will become apparent to those skilled in the art in light of the present disclosure and the accompanying claims.

### Example 1—Spheroid Growth Inhibition

NCI-H1792 and NCI-H23 lung adenocarcinoma (LUAD) cells lines were obtained from ATCC and maintained in RPMI 1640 medium supplemented with 10% fetal bovine serum and penicillin-streptomycin as per ATCC guidelines. Cells were maintained in humidified incubator at 37° C. with 5% CO₂.

Spheroid assays were performed in 96 well ultralow attachment plates (ULA-96U) from Nexcelom & Thermo Fisher Scientific. Two hundred (200) NCI-H1792 or NCI-H23 cells were dispensed in 200 μL of complete growth medium per well (n=10 to 12 wells per treatment group) of each ultralow attachment plate to allow formation of one spheroid per well with a diameter between 200 and 250 μm before the start of treatment (cell seeding numbers were previously optimized so that formed spheroids possessed this desired dimension). To aid spheroid formation, dispensed cells were centrifuged at 220×g for 6 minutes in the ultralow attachment plates and allowed to form compact spheroids for 4 days prior to the initiation of treatment. After spheroids were formed, 150 μL of medium was aspirated from each well without disturbing the spheroid, and fresh RPMI medium of the same volume was added containing single agent compounds (palbociclib, PF-07220060, PF-07104091), or selected combinations thereof). Final concentrations of each compound in the wells were: 30 nM palbociclib; 300 nM PF-07220060 or PF-07104091. DMSO (0.01%) was used as the vehicle control. DMSO and all compounds were diluted in cell medium. Medium and compounds were replenished twice per week, with 3 and 4-day intervals. Replenishment was executed by aspirating 150 μL of medium per well without disturbing the spheroid and then adding the same volume of premixed medium/compound solution to spheroids. Spheroid diameter was quantified immediately following each medium change twice a week (on every 3^{rd} or 4^{th} day) throughout the duration of the assay.

Average diameters of tumor spheroids were plotted in GraphPad Prism 8 and the AUC calculated. AUC baseline was determined by the average tumor spheroid diameter at Day 0 in the vehicle (DMSO) controls. Spheroid growth inhibition, or SGI, was calculated as follows: SGI=(1−AUC treatment/AUC DMSO)×100%. SGI for all treatment arms was derived at the timepoint when the vehicle (DMSO) treated spheroids reach their maximal diameter (usually close to 1 mm but this can differ among cell lines); this corresponds to the last time point taken for the vehicle (DMSO) treated spheroids. SEM was calculated based on n=10 to 12 wells per test group.

Growth of lung adenocarcinoma multicellular tumor spheroids (MCTS) was monitored over time to assess: (i) amplitude of response (SGI) and (ii) duration of response to the CDK4 inhibitor PF-07220060 as a single agent, and in combination with the CDK2 inhibitor PF-07104091.

PF-07220060 and PF-07104091 are projected to attain higher tolerated clinical exposures (due to the reduction of dose-limiting toxicity) than palbociclib in humans (free average plasma concentration of 30 nM). Spheroids were therefore treated with 300 nM of PF-07220060 or PF-07104091 and compared to palbociclib at the clinically relevant concentration of 30 nM. PF-07104091 treatment sensitized NCI-H1792 and NCI-H23 spheroids to PF-07220060, indicating that use of both agents in combination increases the anti-tumor effect when compared to either compound alone. Moreover, 300 nM PF-07220060 plus 300 nM PF-07104091 showed superior spheroid growth inhibition when compared to 30 nM palbociclib plus 300 nM PF-07104091. Data are provided in Table 1.

TABLE 1
Summary of treatments and observed spheroid growth inhibition (SGI)

| Cell line | Treatment | SGI (%) | Days |
|---|---|---|---|
| NCI-H1792 | 30 nM palbociclib | 54 | 14 |
| | 300 nM PF-07220060 | 81 | 14 |
| | 300 nM PF-07104091 | 12 | 14 |
| | 30 nM palbociclib + 300 nM PF-07104091 | 51 | 14 |
| | 300 nM PF-07220060 + 300 nM PF-07104091 | 94 | 14 |
| NCI-H23 | 30 nM palbociclib | 9 | 14 |
| | 300 nM PF-07220060 | 35 | 14 |
| | 300 nM PF-07104091 | 9 | 14 |
| | 30 nM palbociclib + 300 nM PF-07104091 | 31 | 14 |
| | 300 nM PF-07220060 + 300 nM PF-07104091 | 63 | 14 |

### Example 2—Inhibition of Proliferation of ER+ Human Breast Cancer Cells with PF-07104091 in Combination with CDK4 Inhibitor PF-07220060

The combination effects of PF-07104091 and PF-07220060 were evaluated in a cell proliferation assay with MCF7 and T47D BC cell lines. Each compound exhibited potent single agent dose dependent growth inhibition in both cell models.

Drug combinations were analyzed using the Dose Equivalence Principle, and the Loewe Volume score. When modeling synergy using the Dose Equivalence Principle, the Loewe Volume score for the combination compounds should be greater than the compound self-cross controls, and the Combination Index (C) score for the combination compounds should be less than compound self-cross controls. The combinations of PF-07104091 with PF-07220060 demonstrated favorable profiles for these two metrics, Loewe Volume and CI, as compared to the self-cross controls in the MCF7 breast cancer cell line (Table 2) and the T47D breast cancer cell line (Table 3). The isobolograms set to an anti-proliferation threshold at 70% confirmed a synergistic response. Based on the Loewe Volumes, CI values, and isobolograms, modest combination synergy was observed in these cell lines with the highest combinatorial benefit seen in the MCF7 model.

TABLE 2
Inhibition of Proliferation of ER+ Human Breast Cancer Cells
with PF-07104091 in Combination with PF-07220060 in MCF7 Cells

| | | | Best | Best | | Loewe | |
|---|---|---|---|---|---|---|---|
| | Combination | Best | CI | CI | Loewe | Volume | |
| Description | name | CI | error | Level | Volume | error | N |
| Combination | PF-07104091 × | 0.75 | 0.02 | 70 | 0.74 | 0.02 | 8 |
| | PF-07220060 | | | | | | |
| Self-cross | PF-07104091 × | 1.04 | 0.03 | 70 | −0.08 | 0.08 | 4 |
| control | PF-07104091 | | | | | | |
| | PF-07220060 × | 1.06 | 0.01 | 70 | −0.02 | 0.04 | 2 |
| | PF-07220060 | | | | | | |

TABLE 3
Inhibition of Proliferation of ER+ Human Breast Cancer Cells
with PF-07104091 in Combination with PF-07220060 in T-47D Cells

| | | | Best | Best | | Loewe | |
|---|---|---|---|---|---|---|---|
| | | Best | CI | CI | Loewe | Volume | |
| Description | Combination | CI | error | Level | Volume | error | N |
| Combination | PF-07104091 × | 0.91 | 0.01 | 70 | 0.54 | 0.02 | 8 |
| | PF-07220060 | | | | | | |
| Self-cross | PF-07104091 × | 0.95 | 0.03 | 70 | 0.01 | 0.05 | 4 |
| control | PF-07104091 | | | | | | |
| | PF-07220060 × | 0.97 | 0.12 | 70 | 0.00 | 0.06 | 2 |
| | PF-07220060 | | | | | | |

### Example 3—Antitumor Efficacy of PF-07104091 in Combination with PF-07220060 in Breast Cancer Tumor Models

### Methods

### In Vivo Efficacy Evaluation and Statistical Analysis

Female NSG mice (Jackson Lab) were subcutaneously implanted with fragment (27 mm³ to 64 mm³ size) into the dorsal region. All mice for T47D, HCC1428 and ST941 PBR studies were supplemented with 8.5 μg/mL estradiol water (β-Estradiol Sigma-Aldrich, cat #E2758-5G) and gave ad libitum till end of study. For the in vivo efficacy studies, tumor volume and body weights were measured twice a week. Tumor volume was calculated using the [(Length×Width×Width)/2)] formula. TGI was calculated as 100*(1−ΔT/ΔC). The ΔC (ΔT) was obtained by subtracting the mean tumor burden in the vehicle (treated) group on the first day of treatment (Day 0) from the mean tumor burden in vehicle (treated) group on the assessment day. Statistical analysis was performed using ANCOVA when the mean tumor volume in vehicle treated mice reached tumor cutoff size.

In Vivo Efficacy Evaluation of PF-07104091 in Combination with PF-07220060 or Palbociclib in the HR+/HER2− T47D Breast Cancer Model

The T47D model was established by implanting passage 3 tumor fragments into recipient mice. To establish the T47D donor mice, tumor cells (5×10⁶ cell/mouse with 50% Cultrex® Basement Membrane Matrix) were subcutaneously implanted in female NSG mice. Once reaching a range of 700 to 800 mm³, donor tumors were subsequently transplanted into secondary recipient mice for a study expansion. When tumor volume reached a range between 101 mm³ to 255 mm³, the tumor bearing mice were randomly assigned to groups (n=8 per group) and dosed with 1) vehicle (0.5% MC with 0.1% Tween 80 in water); 2) PF-07104091 at 150 mg/kg; 3) PF-07220060 at 60 mg/kg; 4) PD-0332991 at 10 mg/kg; 5) PF-07104091 at 150 mg/kg plus PF-07220060 at 60 mg/kg PF; 6) PF-07104091 at 150 mg/kg plus palbociclib at 10 mg/kg. PF-07104091 (lot 016), PF-07220060 (lot 019) and PD-0332991 (lot GR08498) were administered (PO.) BID (7 hr apart). All mice received treatment continuously until Day 41. TGI was assessed on Day 41 post first dose.

In Vivo Efficacy Evaluation of PF-07104091 in Combination with PF-07220060 or Palbociclib in the HR+/HER2− HCC1428 Breast Cancer Model

The HCC1428 model was established by implanting passage 4 tumor fragments into recipient mice. To establish the HCC1428 donor mice, tumor cells (5×10⁶ cell/mouse with 50% Cultrex® Basement Membrane Matrix) were subcutaneously implanted in female NSG mice. Once reaching a range of 700 to 800 mm³, donor tumors were subsequently transplanted into secondary recipient mice for a study expansion. When tumor volume reached a range between 100 mm³ to 272 mm³, the tumor bearing mice were randomly assigned to groups (n=10 per group) and dosed 1) vehicle (0.5% MC with 0.1% Tween 80 in water); 2) PF-07104091 at 150 mg/kg; 3) PF-07220060 at 60 mg/kg; 4) PD-0332991 at 10 mg/kg; 5) PF-07104091 at 150 mg/kg plus PF-07220060 at 60 mg/kg PF; 6) PF-07104091 at 150 mg/kg plus palbociclib at 10 mg/kg. PF-07104091 (lot 016), PF-07220060 (lot 019) and PD-0332991 (lot GR08498) were administered (PO.) BID (7 hr apart). All mice received treatment continuously until Day 42. TGI was assessed on Day 42 post first dose.

### Establishment of the In Vivo Palbociclib-Acquired Resistance HR+/HER2− BC PDX Model, ST941 PBR

The ST941 PBR palbociclib resistant model was obtained from XENOSTART™ LLC, San Antonio, Texas. We established model in house by continuous treatment of the ST941 PBR tumor bearing mice with PD-0332991 at 50 mg/kg, PO QD plus fulvestrant at 10 mg/kg, SC twice in the first week, then weekly thereafter. To propagate donors for the TGI study, tumors were transplanted to recipient mice. One week after the re-implantation, the recipient mice received continuous treatment with palbociclib plus fulvestrant (dosing schedule described above). After 1 to 2 serial in vivo propagation with the same treatment regimen, the resistant tumors in the range of 700 to 800 mm³ were re-implanted for a study expansion. The remaining tumor fragments were viably frozen for future use.

To confirm resistance to treatment, ST941 PBR tumor bearing mice (p12) received palbociclib (50 mg/kg) plus fulvestrant (10 mg/kg) using the same treatment schedule as described above.

In Vivo Efficacy Evaluation of PF-07104091 in Combination with PF-07220060 or Palbociclib in the ST941PBR PDX Model

The live donor mice were established by implanting ST941 PBR (p11) tumor fragments. Once reaching a range of 700 to 800 mm³, the donor tumors were subsequently transplanted into secondary recipient mice for a study expansion. To maintain the resistant clones, the ST941 PBR tumor bearing mice were treated with PD-0332991 at 50 mg/kg, PO. QD plus fulvestrant at 10 mg/kg, SC twice in the first week, then weekly afterwards for 10 weeks. The treatment was initiated one week after the implant until day of randomization for study enrollment. When tumor volume reached a range between 121 mm³ to 228 mm³, the tumor bearing mice were randomly assigned to groups (n=10 per groups) and dosed with 1) vehicle (0.5% MC with 0.1% Tween 80 in water); 2) PF-07104091 at 150 mg/kg; 3) PF-07220060 at 60 mg/kg; 4) PD-0332991 at 10 mg/kg; 5) PF-07104091 at 150 mg/kg plus PF-07220060 at 60 mg/kg PF; 6) PF-07104091 at 150 mg/kg plus palbociclib at 10 mg/kg; 7) PD-0332991 at 10 mg/kg plus fulvestrant at 10 mg/kg; 8) PD-0332991 at 50 mg/kg plus fulvestrant at 10 mg/kg. PF-07104091 (lot 016), PF-07220060 (lot 019), PF-06873600 (lot 022) and PD-0332991 (lot GR08498) at 10 mg/kg were administered (PO) BID (7 hr apart); PD-0332991 (lot GR08498) at 50 mg/kg were administered (PO) QD and Fulvestrant at 10 mg/kg, SC twice in the first week, then weekly afterwards. All mice received treatment continuously until Day 28. TGI was assessed on Day 28 post first dose.

### Results and Discussions

PF-07104091 was also evaluated as a single agent and in combination with palbociclib or PF-07220060 in three HR+, HER2− BC models: T47D, HCC1428, and palbociclib-resistant ST941 PBR PDX. Treatments were administered PO, BID at mg/kg (mpk) dose indicated in Table 4 (except the 50 mg/kg QD palbociclib group and SC fulvestrant twice the first week and weekly thereafter). There were no significant body weight changes or other clinical observations noted throughout the treatment period in 10 any of the 3 models (clinical pathology not conducted).

In the T47D model, PF-07104091, PF-07220060, and palbociclib single agent treatment showed significant TGI efficacy versus vehicle (p<0.05). PF-07104091 plus PF-07220060 treatment displayed significantly enhanced efficacy (TGI 106%) versus either PF-07220060 (TGI 73%) or PF-07104091 (TGI 82%) monotherapies (p<0.05 vs 15 either of the single agents). PF-07104091 plus palbociclib (TGI 100%) also displayed significantly enhanced efficacy versus either monotherapy (p<0.05 vs either of the single agents).

In the HCC1428 model, each of PF-07104091, PF-07220060, and palbociclib single agent treatment also showed significant TGI efficacy versus vehicle (p<0.05). 20 PF-07104091 plus PF-07220060 treatment displayed significantly enhanced efficacy (TGI 110%) versus either PF-07220060 (TGI 89%) or PF-07104091 (TGI 95%) monotherapies (Table 4).

In the palbociclib resistant ST941 PBR PDX model, palbociclib (10 mg/kg BID or 50 mg/kg QD) treatment in combination with fulvestrant showed significant but minimum response (30% and 26% TGI, respectively, versus vehicle), confirming acquired resistance (Table 4). PF-07104091 in combination with palbociclib resulted in significant TGI (49%) versus vehicle or either PF-07104091 (TGI 34%) or palbociclib monotherapies. PF-07104091 treatment in combination with PF-07220060 significantly enhanced efficacy versus either monotherapy (combination TGI of 98% versus 34% for PF-07104091 monotherapy and 28% for PF-07220060 monotherapy). The combination of PF-07104091 plus PF-07220060 resulted in significantly improved benefit in comparison to the combination of PF-07104091 plus palbociclib in this palbociclib resistant ST941PBRPDX model.

TABLE 4
PF-07104091 Activity as a Single Agent or In Combination with
PF-07220060 or Palbociclib in HR+, HER2- Breast Cancer Models

| | | T47D | HCC1428 | ST941PBR |
|---|---|---|---|---|
| | | Day 41 | Day 42 | Day 28 |
| Group | Treatment | TGI % | TGI % | TGI % |
| 1 | Vehicle | | | |
| 2 | PF-07104091 150 mpk BID | 82^{*} | 95^{*} | 24^{*} |
| 3 | PF-07220060 60 mpk BID | 73^{*} | 89^{*} | 28^{*} |
| 4 | Palbociclib 10 mpk BID | 72^{*} | 44^{*} | 15 |
| 5 | PF-07104091 150 mpk BID + | 106^{*a,b} | 110^{*a,b} | 98^{*a,b} |
| | PF-07220060 60 mpk BID | | | |
| 6 | PF-07104091 150 mpk BID + | 100^{*a,c} | 106^{*a,c} | 49^{*a,c} |
| | Palbociclib 10 mpk BID | | | |
| 7 | Palbociclib 10 mpk BID + | NA | NA | 30^{*} |
| | Fulvestrant 10 mpk (2x week 1, | | | |
| | then Q7Dx4) | | | |
| 8 | Palbociclib 50 mpk QD + | NA | NA | 26^{*} |
| | Fulvestrant 10 mpk (2x week, | | | |
| | then Q7Dx4) | | | |

TGI was assessed post first dose on Day 41 for T47D (n = 7 to 8/group), Day 42 for HCC1428 (n = 10/group), and Day 28 for ST941PBR (n = 9 to 10/group). Vehicle = 0.5% MC with 0.1% Tween 80 in water.
Statistical analysis was performed using ANCOVA.
*indicates p < 0.05 vs. vehicle; ^{a}indicates p < 0.05 vs. PF-07104091 treatment; ^{b}indicates p < 0.05 vs PF-07220060; ^{c}indicates p < 0.05 vs palbociclib.

### Example 4—Antitumor Efficacy of Low Dose PF-07104091 in Combination with PF-07220060 in Breast Cancer Tumor Models

### Methods

### In Vivo Efficacy Evaluation and Statistical Analysis

Female NSG mice (Jackson Lab) were subcutaneously implanted with fragment (27 mm³ to 64 mm³ size) into the dorsal region. All mice for HCC1428, MCF7 and ST941 PBR studies were supplemented with 8.5 μg/mL estradiol water (β-Estradiol Sigma-Aldrich, cat #E2758-5G) and gave ad libitum till end of study. For the in vivo efficacy studies, tumor volume and body weights were measured twice a week. Tumor volume was calculated using the [(Length×Width×Width)/2)] formula. TGI was calculated as 100*(1−ΔT/ΔC). The ΔC (ΔT) was obtained by subtracting the mean tumor burden in the vehicle (treated) group on the first day of treatment (Day 0) from the mean tumor burden in vehicle (treated) group on the assessment day. Statistical analysis was performed using ANCOVA when the mean tumor volume in vehicle treated mice reached tumor cutoff size.

In Vivo Efficacy Evaluation of PF-07104091 in Combination with PF-07220060 or Palbociclib in the HR+/HER2− HCC1428 Breast Cancer Model

The HCC1428 model was established by implanting passage 1 tumor fragments into recipient mice. To establish the HCC1428 donor mice, tumor cells (5×10⁶ cell/mouse with 50% Cultrex® Basement Membrane Matrix) were subcutaneously implanted in female NSG mice. Once reaching a range of 700 to 800 mm³, donor tumors were subsequently transplanted into secondary recipient mice for a study expansion. When tumor volume reached a range between 119 mm³ to 267 mm³, the tumor bearing mice were randomly assigned to groups (n=10 per group) and dosed with 1) vehicle (0.5% MC with 0.1% Tween 80 in water); 2) PF-07104091 at 120 mg/kg; 3) PF-07220060 at 60 mg/kg; 4) PF-06873600 at 30 mg/kg; 5) Palbociclib at 10 mg/kg plus fulvestrant at 10 mg/kg; 6) PF-07104091 at 120 mg/kg plus PF-07220060 at 20 mg/kg; 7) PF-07104091 at 75 mg/kg plus PF-07220060 at 40 mg/kg; 8) PF-07104091 at 35 mg/kg plus PF-07220060 at 60 mg/kg. PF-07104091 (lot 022), PF-07220060 (lot CPo126812-01-SY-5700-01), PF-06873600 (lot 022) and PD-0332991 (lot GR08498) were administered (PO) BID (7 hr apart) while fulvestrant (lot 20160224-2) at 10 mg/kg, SC twice in the first week, then weekly afterwards. All mice received treatment continuously until Day 42. TGI was assessed on Day 42 post first dose.

In Vivo Efficacy Evaluation of PF-07104091 in Combination with PF-07220060 or Palbociclib in the HR+/HER2− MCF7 Breast Cancer Model

The MCF7 model was established by implanting donor tumor fragments into recipient mice. To establish the MCF7 donor mice, tumor cells (5×10⁶ cell/mouse with 50% Cultrex® Basement Membrane Matrix) were subcutaneously implanted in female NSG mice. Once reaching a range of 700 to 800 mm³, donor tumors were subsequently transplanted into secondary recipient mice for a study expansion. When tumor volume reached a range between 141 mm³ to 231 mm³, the tumor bearing mice were randomly assigned to groups (n=10 per group) and dosed with 1) vehicle (0.5% MC with 0.1% Tween 80 in water); 2) PF-07104091 at 120 mg/kg; 3) PF-07220060 at 60 mg/kg; 4) PF-06873600 at 30 mg/kg; 5) Palbociclib at 10 mg/kg plus fulvestrant at 10 mg/kg; 6) PF-07104091 at 120 mg/kg plus PF-07220060 at 20 mg/kg; 7) PF-07104091 at 75 mg/kg plus PF-07220060 at 40 mg/kg; 8) PF-07104091 at 35 mg/kg plus PF-07220060 at 60 mg/kg. PF-07104091 (lot 022), PF-07220060 (lot CPo126812-01-SY-5700-01), PF-06873600 (lot 022) and PD-0332991 (lot GR08498) were administered (PO) BID (7 hr apart) while fulvestrant (lot 20160224-2) at 10 mg/kg, SC twice in the first week, then weekly afterwards. All mice received treatment continuously until Day 29. TGI was assessed on Day 29 post first dose

### Establishment of the In Vivo Palbociclib-Acquired Resistance HR+/HER2− BC PDX Model, ST941 PBR

The ST941 PBR palbociclib resistant model was obtained from XENOSTART™ LLC, San Antonio, Texas. We established model in house by continuous treatment of the ST941 PBR tumor bearing mice with PD-0332991 at 50 mg/kg, PO QD plus fulvestrant at 10 mg/kg, SC twice in the first week, then weekly thereafter. To propagate donors for the TGI study, tumors were transplanted to recipient mice. One week after the re-implantation, the recipient mice received continuous treatment with palbociclib plus fulvestrant (dosing schedule described above). After 1 to 2 serial in vivo propagation with the same treatment regimen, the resistant tumors in the range of 700 to 800 mm³ were re-implanted for a study expansion. The remaining tumor fragments were viably frozen for future use.

To confirm resistance to treatment, ST941 PBR tumor bearing mice (p14) received palbociclib (50 mg/kg) plus fulvestrant (10 mg/kg) using the same treatment schedule as described above.

In Vivo Efficacy Evaluation of PF-07104091 in Combination with PF-07220060 or Palbociclib in the ST941PBR PDX Model

The live donor mice were established by implanting ST941 PBR (p13) tumor fragments. Once reaching a range of 700 to 800 mm³, the donor tumors were subsequently transplanted into secondary recipient mice for a study expansion. To maintain the resistant clones, the ST941 PBR tumor bearing mice were treated with PD-0332991 at 50 mg/kg, PO. QD plus fulvestrant at 10 mg/kg, SC twice in the first week, then weekly afterwards for 10 weeks. The treatment was initiated one week after the implant until day of randomization for study enrollment. When tumor volume reached a range between 137 mm³ to 216 mm³, the tumor bearing mice were randomly assigned to groups (n=10 per group) and dosed with 1) vehicle (0.5% MC with 0.1% Tween 80 in water); 2) PF-07104091 at 120 mg/kg; 3) PF-07220060 at 60 mg/kg; 4) PF-06873600 at 30 mg/kg; 5) Palbociclib at 10 mg/kg plus fulvestrant at 10 mg/kg; 6) PF-07104091 at 120 mg/kg plus PF-07220060 at 20 mg/kg; 7) PF-07104091 at 75 mg/kg plus PF-07220060 at 40 mg/kg; 8) PF-07104091 at 35 mg/kg plus PF-07220060 at 60 mg/kg. PF-07104091 (lot 021), PF-07220060 (lot CPo126812-01-SY-5700-01), PF-06873600 (lot 022) and PD-0332991 (lot GR08498) were administered (PO) BID (7 hr apart) while fulvestrant (lot 20160224-2) at 10 mg/kg, SC twice in the first week, then weekly afterwards. All mice received treatment continuously until Day 28. TGI was assessed on Day 28 post first dose.

### Results and Discussions

Lower dose levels of PF-07104091+PF-07220060 were also evaluated to test if combination could improve efficacy versus monotherapy, PF-06873600 and SOC palbociclib+fulvestrant in three HR+, HER2− BC models: MCF7, HCC1428, and palbociclib-resistant ST941 PBR PDX. Treatments were administered PO, BID at mg/kg (mpk) dose indicated in Table 5 (except the 10 mg/kg SC fulvestrant twice the first week and weekly thereafter). There were no significant body weight changes or other clinical observations noted throughout the treatment period in any of the 3 models (clinical pathology not conducted).

In the MCF7 model, SOC palbociclib 10 mg/kg+fulvestrant 10 mg/kg and PF-07104091 at 120 mg/kg, PF-07220060 at 60 mg/kg, PF-06873600 at 30 mg/kg single agent treatment showed significant TGI efficacy versus vehicle (TGI 84%, 60%, 79% and 70%, respectively; p<0.05). Low dose of PF-07104091+PF-07220060 treatment groups—PF-4091 35 mg/kg+PF-0060 60 mg/kg, PF4091 75 mg/kg+PF-0060 40 mg/kg and PF-4091 120 mg/kg+PF0060 20 mg/kg showed significant regression (TGI 106%, 106%, 105%) in comparison to vehicle, SOC palbociclib 10 mg/kg+fulvestrant 10 mg/kg and single agent treatment of PF-07104091 at 120 mg/kg, PF-07220060 at 60 mg/kg and PF-06873600 at 30 mg/kg. There was also no significant efficacy difference between the low dose PF-4091+PF-0060 groups (p>0.05) in MCF7 model.

In the HCC1428 model, SOC palbociclib 10 mg/kg+fulvestrant 10 mg/kg and PF-07104091 at 120 mg/kg, PF-07220060 at 60 mg/kg, PF-06873600 at 30 mg/kg single agent treatment showed significant TGI efficacy versus vehicle (TGI 70%, 79%, 70% and 97%, respectively; p<0.05). Low dose of PF-07104091+PF-07220060 treatment groups—PF-4091 35 mg/kg+PF-0060 60 mg/kg, PF4091 75 mg/kg+PF-0060 40 mg/kg and PF-4091 120 mg/kg+PF0060 20 mg/kg showed significant regression (TGI 110%, 110%, 109%, respectively) in comparison to vehicle, SOC palbociclib 10 mg/kg+fulvestrant 10 mg/kg and single agent treatment of PF-07104091 at 120 mg/kg, PF-07220060 at 60 mg/kg and PF-06873600 at 30 mg/kg. There was also no significant efficacy difference between the low dose PF-4091+PF-0060 groups (p>0.05) in HCC1428 model.

In the palbociclib resistant ST941 PBR PDX models, palbociclib (10 mg/kg BID) treatment in combination with fulvestrant showed minimum response (14% TGI versus vehicle), confirming acquired resistance (Table 5). PF-07220060 at 60 mg/kg showed minimum response (TGI 19%, p>0.05) and PF-07104091 at 120 mg/kg and PF-0873600 at 30 mg/kg showed significant but moderate efficacy versus vehicle (TGI 36% and 47%, respectively; p<0.05). Low dose of PF-07104091+PF-07220060 treatment groups—PF-4091 35 mg/kg+PF-0060 60 mg/kg, PF4091 75 mg/kg+PF-0060 40 mg/kg and PF-4091 120 mg/kg+PF-0060 20 mg/kg showed significant inhibition (TGI 87%, 98%, 94%, respectively) in comparison to vehicle, SOC palbociclib 10 mg/kg+fulvestrant 10 mg/kg and single agent treatment of PF-07104091 at 120 mg/kg, PF-07220060 at 60 mg/kg and PF-06873600 at 30 mg/kg. Between the low dose groups, there was no significant efficacy difference between PF-4091 75 mg/kg+PF-0060 40 mg/kg and PF-4091 120 mg/kg+PF-0060 20 mg/kg but significantly different between PF-4091 75 mg/kg+PF-0060 40 mg/kg and PF-4091 120 mg/kg+PF-0060 20 mg/kg versus PF-4091 35 mg/kg+PF-0060 60 mg/kg (p<0.05).

Results demonstrate the ability to lower exposures of either agent, or both, and maintain maximal tumor control (regressions on study) in the cell line models of primary disease (MCF7 & HCC1428). In the model of therapy resistance, STR941-PBR, lowering the CDK2i dose in the combo regimen to 35 mg BID did differentiate from the other combo groups tested, demonstrating slightly less TGI on study and a more robust recovery from treatment

TABLE 5
Low dose PF-07104091 Activity In Combination with
PF-07220060 in HR+, HER2-Breast Cancer Models

| | | MCF7 | HCC1428 | ST941PBR |
|---|---|---|---|---|
| | | Day 29 | Day 42 | Day 28 |
| Group | Treatment | TGI % | TGI % | TGI % |
| 1 | Vehicle | | | |
| 2 | PF-07104091 120 mpk BID | 60^{*} | 79^{*} | 36^{*} |
| 3 | PF-07220060 60 mpk BID | 79^{*} | 70^{*} | 19 |
| 4 | PF-06873600 30 mpk BID | 70^{*} | 97^{*} | 47^{*} |
| 5 | Palbociclib 10 mpk BID + | 84^{*} | 70^{*} | 14 |
| | Fulvestrant 10 mpk (2x | | | |
| | week 1, then Q7D) | | | |
| 6 | PF-07104091 120 mpk BID + | 105^{*a,b} | 109^{*a,b} | 94^{*a,b,c} |
| | PF-07220060 20 mpk BID | | | |
| 7 | PF-07104091 75 mpk BID + | 106^{*a,b} | 110^{*a,b} | 98^{*a,b,c} |
| | PF-07220060 40 mpk BID | | | |
| 8 | PF-07104091 35 mpk BID + | 106^{*a,b} | 110^{*a,b} | 87^{*a,b} |
| | PF-07220060 60 mpk BID | | | |

TGI was assessed post first dose on Day 29 for MCF7 (n = 10/group), Day 42 for HCC1428 (n = 10/group), and Day 28 for ST941PBR (n = 9 to 10/group). Vehicle = 0.5% MC with 0.1% Tween 80 in water.
Statistical analysis was performed using ANCOVA.
*indicates p < 0.05 vs vehicle; ^{a}indicates p < 0.05 vs monotherapy (PF-07104091, PF-07220060, PF-06873600); ^{b}indicates p < 0.05 vs palbociclib + fulv; ^{c}indicates p < 0.05 vs PF-4091 35 mpk + PF-0060 60 mpk.

### Example 5—Combinatorial Activity of PF-07104091 and PF-07220060 in Lung Adenocarcinomas

Combinatorial activity of PF-07104091 and PF-07220060 was assessed in LUADs as single agents (s.a.) or in combination. Data are provided in Table 6.

TABLE 6

| | | | | PF-0060 | |
|---|---|---|---|---|---|
| | | | | (111-1000 nM) | |
| | Molecular | PF-0060 | PF-4091 | and PF-4091 | |
| Cell Line | Feature | (nM) s.a. | (nM) s.a. | (150-450 nM) | |
| H23 | KRAS G12C | 285 | NA | S | (95%) |
| H1792 | KRAS G12C | 210 | NA | CDK2 not active | |
| H358 | KRAS G12C | 120 | NA | A-S | (96%) |
| H2030 | KRAS G12C | 950 | NA | S | (90%) |
| HCC44 (RB-) | KRAS G12C | >1000 | NA | CDK4 not active | |
| SW1573 | KRAS G12C | >1000 | NA | S | (92%) |
| H1373 | KRAS G12C | 470 | NA | S | (93%) |
| NCI-2291 | KRAS | 138 | ~1200 | A | (82%) |
| | G12C/F/V | | | | |
| A427 | KRAS G12D | 123.8 | 275.6 | A-S | (98%) |
| NCIH1944 | KRAS G13D | 398 | 227 | S | (92%) |
| NCI-H2347 | KRAS L19F | 70 | 364 | A-S | (88%) |
| A549 | WT | 813.8 | 574.5 | A-S | (94%) |
| ABC1 | WT | 407.5 | 716.1 | A | (70%) |
| CAL12T | WT | 210 | 220 | S | (93%) |
| CALU6 | WT | 154.7 | 268.3 | A | (96%) |
| HCC2935 | WT | 141.2 | 280.3 | A | (59%) |
| NCIH2023 | WT | 310 | >450 | A | (91%) |
| NCIH2126 | WT | 255 | 372 | A-S | (93%) |
| NCIH2228 (RB | WT | 839 | ~1000 | A-S | (76%) |

mut)

PF-0060: PF-07220060, PF-4091: PF-0724091
Bliss: A additive, S synergistic; % inh.: maximum combo inh. at highest doses of both cpds; WT: negative for EGFR, ALK, ROS, BRAF, NTRK1 and KRAS

### Example 6—Preliminary Drug-Drug Interaction (DDI) Risk Assessment

Preliminary substrate/victim DDI risk assessment of PF-07104091 was conducted using a static mechanistic model with PF-07220060 at clinically relevant concentrations. (Fahmi O A, Hurst S, Plowchalk D, et al. Comparison of different algorithms for predicting clinical drug-drug interactions, based on the use of CYP3A4 in vitro data: predictions of compounds as precipitants of interaction. Drug Metab Dispos 2009; 37(8):1658-66.) Based on the potential combination doses (300 mg of PF-07220060 with 75 mg of PF-07104091), PF-07220060 was predicted to exhibit a potential perpetrator based cytochrome P450 3A (CYP3A) time dependent inhibition (TDI) on PF-07104091 in combination, resulting in a AUG ratio (AUCR) of ˜2.

Beyond the potential CYP3A DDI, there is no additional data which indicates a significant DDI risk between PF-07104091 and PF-07220060 both as perpetrator and substrate/victim at clinically relevant concentrations as well as the addition of letrozole or fulvestrant to this combination.

Example 7—Phase 1 b/2, Open-Label, Multicenter, Nonrandomized, Dose Escalation and Dose Expansion Study to Evaluate the Safety, Tolerability, PK, PD, and Antitumor Activity of PF-07220060 Administered in Combination with PF-07104091

A Phase 1 b/2, open-label, multicenter, nonrandomized, dose escalation and dose expansion study to evaluate the safety, tolerability, PK, PD, and antitumor activity of PF-07220060 and PF-07104091 (doublet combination) in participants with metastatic breast cancer (mBC) or other advanced solid tumors (Part 1) and when the doublet combination is administered with endocrine therapy (ET) in participants with metastatic/advanced mBC (Part 2). 12 participants have received the doublet combination therapy in Part 1 (dose escalation) across 4 dose cohorts as of data cut-off date.

In Part 2, a safety run-in of approximately 6 participants will be enrolled to each of the 2 triplet combinations (PF-07220060, PF-07104091, and fulvestrant, or PF-07220060, PF-07104091, and letrozole), to establish safety and tolerability of the combinations. Part 2A will include participants who were previously treated with a CDK4/6 inhibitor and these participants will receive a triplet of PF-07220060, PF-07104091, and fulvestrant. Part 2B will include participants who are treatment naïve to CDK4/6 inhibitors but have been previously treated with 1 ET, and these participants will receive a triplet of PF-07220060, PF-07104091, and fulvestrant. Part 2C will include participants who are treatment naïve to CDK4/6 inhibitors and ET for advanced disease, and these participants will receive a triplet of PF-07220060, PF-07104091, and letrozole.

Dose escalation of PF-07220060 and PF-07104091 will continue in a stepwise manner according to Bayesian logistic regression model (BLRM) recommendation. Dose escalation will continue until the MTD of PF-07220060 and PF-07104091 combination is determined or stopping criteria for further escalation is met. The highest dose of PF-07220060 and PF-07104091 to be tested in this study will not be higher than the monotherapy MTD/Recommended dose for expansion (RDE) of each drug as determined from single agent studies, respectively. Dose escalation decisions will also take into consideration all available clinical data from this study.

Enriched PK sampling of both PF-07220060 and PF-07104091 will be performed to evaluate the PK and DDI potential. To assess potential DDI, participants in Part 1 will be administered a single dose of PF-07104091 on Day −1 with PK assessment up to 24 hour postdose of PF-07104091. Beginning, Cycle 1 Day1 participants will receive both PF-07220060 and PF-07104091 on a continuous schedule given as BID regimen and PK assessment after multiple doses will be evaluated on Cycle 1 Day 15. The PK assessments may be modified (e.g., removal of Cycle 1 Day-1 evaluation) based on the evolving data from initial cohorts on the DDI assessment.

Part 1 will enroll participants with metastatic/advanced breast cancer and other advanced or metastatic solid tumors with no standard alternative treatment option to determine the doublet MTD/RDE. Each cohort will enroll approximately 2 to 4 participants. At least 6 participants will be treated at the recommended combination doses of PF-07220060 and PF-07104091 prior to start of dose expansion.

Participants with BC may also receive fulvestrant after at least 2 cycles of the combination of PF-07220060 and PF-07104091 at the discretion of the investigator and with the approval of the sponsor if:
the current dose of PF-07220060 and PF-07104091 received by the participants as combination therapy has been determined to be safe by BLRM;
the participant has not experienced an adverse event (AE) meeting DLT criteria, or;
an AE meeting DLT criteria has resolved and the participant has resumed PF-07220060 and PF-07104091 combination therapy for at least 28 days without an AE meeting DLT criteria.

Part 2 (dose expansion) will enroll up to 3 cohorts of participants with advanced or metastatic HR-positive HER2-negative BC to be treated with the triplet combination of PF-07220060, PF-07104091, and ET (letrozole or fulvestrant).
Part 2A will include participants who were previously treated with CDK4/6 inhibitors.
Part 2B will include participants who are naïve to CDK4/6 inhibitors but who were previously treated with 1 ET.
Part 2C will include participants who are treatment naïve to CDK4/6 inhibitors and ET for advanced disease.

Participants in Part 2A and 2B will be treated with a triplet of PF-07220060, PF-07104091, and fulvestrant beginning at Cycle 1 and participants in Part 2C will be treated with a triplet of PF-07220060, PF-07104091, and letrozole beginning at Cycle 1.

A dose escalation or de-escalation approach at full or intermediate dose levels of PF-07220060 or PF-07104091, depending on emerging clinical data, will be utilized to select combination RDE. BLRM along with the EWOC criteria will be used in Part 1 to determine the MTDs.

All cycles are 28 days in length and treatment will continue until progression of disease, uncontrollable toxicity, a decision by the participant or investigator to discontinue treatment or the study is terminated. Participants experiencing toxicity including a DLT may be managed with dose modification or discontinuation from treatment of either PF-07220060 or PF-07104091, or both.

PF-07220060 and PF-07104091 will be administered orally BID continuous daily dosing. To enable the optimal dosing regimen of PF-07220060 and PF-07104091 in combination with an endocrine therapy, i.e., letrozole or fulvestrant, an alternative dosing regimen(s), schedule(s), and PK timepoints may be considered based on emerging PK data, safety, tolerability, laboratory, and PD.

As of the data cut-off date, a total of 12 participants have received PF-07220060 in combination with PF-07104091 (selective CDK2 inhibitor) in Part 1 (dose escalation) across 4 dose cohorts.

### Demographics

Overall, a total of 12 participants (11 females and 1 male) with a mean age of 55.25 years (SD: 11.19) have received PF-07220060+PF-07104091. The participants had advanced cancers including HR+ HER2− breast cancer (n=8), small cell cancer (n=1), ovarian (n=1), small cell lung cancer (n=1), and not yet reported (n=1).

### Treatment-Emergent All-Causality Adverse Events

Of the 12 treated participants, 10 (83.3%) participants reported a total of 88 Treatment-emergent adverse events (TEAEs).

Most frequently reported (20%) all-causality were nausea (58.3%), diarrhoea, neutrophil count decreased and white blood cell count decreased (50.0% each), anaemia (41.7%), lymphocyte count decreased (33.3%), fatigue, platelet count decreased and vomiting (25.0% each).

No Grade 4 TEAEs were reported. One Grade 5 TEAE of disease progression was reported during the study.

### Treatment Emergent Treatment-Related Adverse Events

A total of 10 (83.3%) of the 12 participants reported treatment-related TEAEs.

Most frequently reported (20%) treatment-related AEs were nausea (58.3%), diarrhoea, neutrophil count decreased and white blood cell count decreased (50.0% each), lymphocyte count decreased (33.3%), anaemia, fatigue, platelet count decreased and vomiting (25.0% each).

No Grade 4 or 5 treatment-related TEAEs were reported during the study.

### Dose-Limiting Toxicities

As of the data cut-off date, there was one DLT (Grade 3 fatigue) reported in one participant who received PF-07220060 300 mg BID+PF-07104091 150 mg BID.

### Adverse Events Leading to Discontinuation

No participants discontinued from study drug due to adverse events.

### Serious Adverse Events and Death

No Serious Adverse Events (SAEs) or deaths related to study treatments have been reported. One participant in Part 1 PF-07220060 200 mg BID+PF-07104091 75 mg BID treatment group reported SAE of dyspnoea. Another participant in Part 1 PF-07220060 300 mg BID+PF-07104091 150 mg BID Treatment Group reported SAE of disease progression which resulted in death. Both SAEs were not related to study treatments.

1. A method of treating cancer in a subject in need thereof comprising administering to the subject:
(a) an amount of a compound of Formula (I): 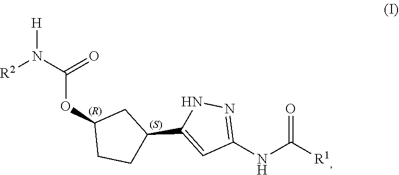
or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) an amount of a compound of Formula (II): 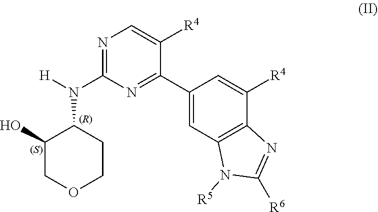
or a pharmaceutically acceptable salt thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl;
wherein the amounts in (a) and (b) together are effective in treating cancer.

2. The method of claim 1, further comprising administering to the subject: (c) an amount of an additional anti-cancer agent; wherein the amounts in (a), (b) and (c) together are effective in treating cancer.

3. The method of claim 1 or 2, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

4. The method of claim 2 or 3, wherein the cancer is hormone receptor positive (HR+) breast cancer and the additional anti-cancer agent is an endocrine therapeutic agent selected from the group consisting of an aromatase inhibitor, a selective estrogen receptor modulator (SERM) and a selective estrogen receptor degrader (SERD).

5. The method of claim 4, wherein the endocrine therapeutic agent is letrozole or fulvestrant.

6. The method of any one of claims 1 to 5, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091) monohydrate.

7. The method of claim 6, wherein the amount of PF-07104091 is from about 75 mg to about 500 mg BID.

8. The method of claim 7, wherein the amount of PF-07104091 is about 75 mg BID, about 150 mg BID, or about 225 mg BID.

9. The method of any one of claims 1 to 8, wherein the compound of Formula (II) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (PF-07220060), or a pharmaceutically acceptable salt thereof.

10. The method of claim 9, wherein the amount of PF-07220060 is from about 100 mg to about 500 mg BID.

11. The method of claim 10, wherein the amount of PF-07220060 is about 100 mg BID, about 200 mg BID, or about 300 mg BID.

12. The method of any one of claims 1 to 11, wherein the cancer is a palbociclib resistant cancer.

13. The method of any one of claims 1 to 11, wherein the subject has previously been treated with a CDK4/6 inhibitor.

14. The method of claim 13, wherein the CDK4/6 inhibitor is palbociclib.

15. A combination comprising:
(a) a compound of Formula (I): 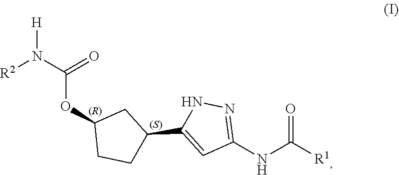
or a pharmaceutically acceptable salt or solvate thereof, wherein:
R¹ is -L-(5-6 membered heteroaryl) or -L-(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂—C₁-C₄ alkyl, where each C₁-C₄ alkyl is optionally substituted by F, OH or C₁-C₄ alkoxy; and
(b) a compound of Formula (II): 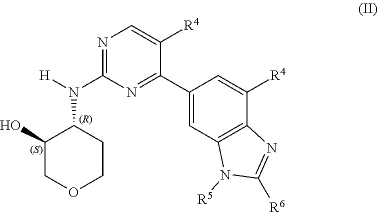
or a pharmaceutically acceptable salt thereof, wherein:
R⁴ is H, F, or Cl;
R⁵ is H, C₁-C₅ alkyl, C₁-C₅ fluoroalkyl or C₃-C₈ cycloalkyl, where each said C₁-C₅ alkyl and C₁-C₅ fluoroalkyl is optionally substituted by R⁸ and each said C₃-C₈ cycloalkyl is optionally substituted by R⁹;
R⁶ is H, C₁-C₄ alkyl or C₁-C₄ fluoroalkyl, where each said C₁-C₄ alkyl and C₁-C₄ fluoroalkyl is optionally substituted by R⁸;
R⁷ is H, F or Cl;
each R⁸ is independently OH, C₁-C₄ alkoxy or NR¹⁰R¹¹;
each R⁹ is independently F, C₁-C₄ alkyl, C₁-C₄ alkoxy, or NR¹⁰R¹¹; and
each R¹⁰ and R¹¹ is independently H or C₁-C₂ alkyl;
wherein the combination of (a) and (b) is effective in treating cancer.

16. The combination of claim 15, further comprising (c) an additional anti-cancer agent; wherein the combination of (a), (b) and (c) is effective in treating cancer.

17. The combination of claim 15 or 16, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

18. The combination of claim 16 or 17, wherein the cancer is hormone receptor positive (HR+) breast cancer and the additional anti-cancer agent is an endocrine therapeutic agent selected from the group consisting of an aromatase inhibitor, a selective estrogen receptor modulator (SERM) and a selective estrogen receptor degrader (SERD).

19. The combination of claim 18, wherein the endocrine therapeutic agent is letrozole or fulvestrant.

20. The combination of any one of claims 15 to 19, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091) monohydrate.

21. The combination of claim 20, wherein the amount of PF-07104091 is from about 50 mg to about 250 mg BID.

22. The combination of claim 21, wherein the amount of PF-07104091 is about 75 mg BID, about 150 mg BID, or about 225 mg BID.

23. The combination of any one of claims 15 to 22, wherein the compound of Formula (II) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (PF-07220060), or a pharmaceutically acceptable salt thereof.

24. The combination of claim 23, wherein the amount of PF-07220060 is from about 100 mg to about 500 mg BID.

25. The combination of claim 24, wherein the amount of PF-07220060 is about 100 mg BID, about 200 mg BID, or about 300 mg BID.
